SEQUENCE LISTING (53 sequences)

SEQ ID NO: 1 - protein, 289 aa, Artificial Sequence
  synthetic polypeptide
MALPVTALLLPLALLLHAARPIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRGGGGSGGGGSGGGGSQVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSSGGGGSGGGGSGGGGSGGGGSAEKDEL

SEQ ID NO: 2 - protein, 348 aa, Artificial Sequence
  synthetic polypeptide
MALPVTALLLPLALLLHAARPIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRGGGGSGGGGSGGGGSQVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSSKPTTTPAPRPPTPAPTIASQPLSLRPEACRPAAGGAVHTRGLDFACDIYIWAPLAGTCGVLLLSLVITLYKYKSRRSFIEEKKMP

SEQ ID NO: 3 - protein, 291 aa, Artificial Sequence
  synthetic polypeptide
MALPVTALLLPLALLLHAARPNIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRGGGGSGGGGSGGGGSQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSGGGGSGGGGSGGGGSGGGGSAEKDEL

SEQ ID NO: 4 - protein, 350 aa, Artificial Sequence
  synthetic polypeptide
MALPVTALLLPLALLLHAARPNIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRGGGGSGGGGSGGGGSQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSKPTTTPAPRPPTPAPTIASQPLSLRPEACRPAAGGAVHTRGLDFACDIYIWAPLAGTCGVLLLSLVITLYKYKSRRSFIEEKKMP

SEQ ID NO: 5 - protein, 488 aa, Artificial Sequence
  synthetic polypeptide
MALPVTALLLPLALLLHAARPNIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRGGGGSGGGGSGGGGSQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 892 bp, Artificial Sequence
  synthetic polynucleotide
gaattcggcttccaccatggctctgcccgtgaccgccctgctgctgcctctggctctgctgctgcacgctgcccgcccaatcgtgatgacccagagcccagccaccctgtccgtgacacctggcgaccgggtgtctctgagctgcagagcctcccagtctatcagcgattacctgcactggtatcagcagaagtcccacgagtctccccggctgctgatcaagtacgctagccagtctatcagcggcatccctagccggttctccggatctggaagcggatccgactttaccctgagcatcaactccgtggagccagaggatgtgggcgtgtactattgccagaatggccactccttccccctgacctttggcgccggcacaaagctggagctgcggagaggcggcggcggctctggaggaggaggaagcggaggaggaggctcccaggtgcagctgcagcagccaggaacagagctggtgcggcccggcagctccgtgaagctgtcctgtaaggcctctggctacaccttcacaagctattgggtgaactgggtgaagcagaggcctgaccagggcctggagtggatcggaaggatcgacccatacgattctgagacacactataaccagaagtttacagacaaggccatcagcaccatcgatacatctagcaataccgcctatatgcagctgtccaccctgacatctgatgccagcgccgtgtactattgttctaggagccctcgcgactcctctacaaatctggcagattggggacagggcaccctggtgacagtgagctccggtggtggcggcagtggtggcggtggctcaggcggtggtggctccggtggcggtggctctgcagaaaaagatgagttgtaactcgag

SEQ ID NO: 7 - DNA, 1069 bp, Artificial Sequence
  synthetic polynucleotide
gaattcggcttccaccatggctctgcccgtgaccgccctgctgctgcctctggctctgctgctgcacgctgcccgcccaatcgtgatgacccagagcccagccaccctgtccgtgacacctggcgaccgggtgtctctgagctgcagagcctcccagtctatcagcgattacctgcactggtatcagcagaagtcccacgagtctccccggctgctgatcaagtacgctagccagtctatcagcggcatccctagccggttctccggatctggaagcggatccgactttaccctgagcatcaactccgtggagccagaggatgtgggcgtgtactattgccagaatggccactccttccccctgacctttggcgccggcacaaagctggagctgcggagaggcggcggcggctctggaggaggaggaagcggaggaggaggctcccaggtgcagctgcagcagccaggaacagagctggtgcggcccggcagctccgtgaagctgtcctgtaaggcctctggctacaccttcacaagctattgggtgaactgggtgaagcagaggcctgaccagggcctggagtggatcggaaggatcgacccatacgattctgagacacactataaccagaagtttacagacaaggccatcagcaccatcgatacatctagcaataccgcctatatgcagctgtccaccctgacatctgatgccagcgccgtgtactattgttctaggagccctcgcgactcctctacaaatctggcagattggggacagggcaccctggtgacagtgagctccaagccaaccacaacccctgcaccaaggccacctacaccagcacctaccatcgcaagccagccactgtccctgaggccagaggcatgtaggcctgcagcaggaggcgccgtgcacacacgcggcctggactttgcctgcgatatctacatctgggcaccactggcaggaacctgtggcgtgctgctgctgagcctggtgattaccctgtataagtacaagtccagacgctcattcattgaggaaaagaaaatgccttaactcgag

SEQ ID NO: 8 - DNA, 898 bp, Artificial Sequence
  synthetic polynucleotide
gaattcggcttccaccatggctctgcccgtgaccgccctgctgctgcctctggctctgctgctgcacgctgcccgcccaaacatcatgatgacccagtcccccagctccctggccgtgtctgccggagagaaggtgaccatgacatgcaagtctagccagtccgtgctgtactcctctaaccagaagaattacctggcctggtatcagcagaagcccggccagagccctaagctgctgatctattgggcaagcacccgggagtccggagtgccagacagattcaccggaagcggatccggaacagacttcaccctgacaatcagctccgtgcagcctgaggacctggccgtgtactattgccaccagtacctgtctagccacaccttcggcggcggcacaaagctggagatcaagaggggaggaggaggatccggaggaggaggctctggcggcggcggcagccagctgcagcagccaggagcagagctggtgaggcccggctcctctgtgaagctgtcttgtaaggccagcggctacaccttcacaaggtattggatccactgggtgaagcagcgccctatccagggcctggagtggatcggcaacatcgacccatctgatagcgagacacactacaatcagaagtttaaggacaaggccaccctgacagtggataagagctccggcaccgcctatatgcagctgtctagcctgacatccgaggactctgccgtgtactattgtgccacagaggatctgtactatgccatggagtactggggccagggcacctccgtgacagtgtcctctggtggtggcggcagtggtggcggtggctcaggcggtggtggctccggtggcggtggctctgcagaaaaagatgagttgtaactcgag

SEQ ID NO: 9 - DNA, 1075 bp, Artificial Sequence
  synthetic polynucleotide
gaattcggcttccaccatggctctgcccgtgaccgccctgctgctgcctctggctctgctgctgcacgctgcccgcccaaacatcatgatgacccagtcccccagctccctggccgtgtctgccggagagaaggtgaccatgacatgcaagtctagccagtccgtgctgtactcctctaaccagaagaattacctggcctggtatcagcagaagcccggccagagccctaagctgctgatctattgggcaagcacccgggagtccggagtgccagacagattcaccggaagcggatccggaacagacttcaccctgacaatcagctccgtgcagcctgaggacctggccgtgtactattgccaccagtacctgtctagccacaccttcggcggcggcacaaagctggagatcaagaggggaggaggaggatccggaggaggaggctctggcggcggcggcagccagctgcagcagccaggagcagagctggtgaggcccggctcctctgtgaagctgtcttgtaaggccagcggctacaccttcacaaggtattggatccactgggtgaagcagcgccctatccagggcctggagtggatcggcaacatcgacccatctgatagcgagacacactacaatcagaagtttaaggacaaggccaccctgacagtggataagagctccggcaccgcctatatgcagctgtctagcctgacatccgaggactctgccgtgtactattgtgccacagaggatctgtactatgccatggagtactggggccagggcacctccgtgacagtgtcctctaagccaaccacaacccctgcaccaaggccacctacaccagcacctaccatcgcaagccagccactgtccctgaggccagaggcatgtaggcctgcagcaggaggcgccgtgcacacacgcggcctggactttgcctgcgatatctacatctgggcaccactggcaggaacctgtggcgtgctgctgctgagcctggtgattaccctgtataagtacaagtccagacgctcattcattgaggaaaagaaaatgccttaactcgag

SEQ ID NO: 10 - DNA, 1549 bp, Artificial Sequence
  synthetic polynucleotide
gaattcggcttccaccatggctctgcccgtgaccgccctgctgctgcctctggctctgctgctgcacgctgcccgcccaaacatcatgatgacccagtcccccagctccctggccgtgtctgccggagagaaggtgaccatgacatgcaagtctagccagtccgtgctgtactcctctaaccagaagaattacctggcctggtatcagcagaagcccggccagagccctaagctgctgatctattgggcaagcacccgggagtccggagtgccagacagattcaccggaagcggatccggaacagacttcaccctgacaatcagctccgtgcagcctgaggacctggccgtgtactattgccaccagtacctgtctagccacaccttcggcggcggcacaaagctggagatcaagaggggaggaggaggatccggaggaggaggctctggcggcggcggcagccagctgcagcagccaggagcagagctggtgaggcccggctcctctgtgaagctgtcttgtaaggccagcggctacaccttcacaaggtattggatccactgggtgaagcagcgccctatccagggcctggagtggatcggcaacatcgacccatctgatagcgagacacactacaatcagaagtttaaggacaaggccaccctgacagtggataagagctccggcaccgcctatatgcagctgtctagcctgacatccgaggactctgccgtgtactattgtgccacagaggatctgtactatgccatggagtactggggccagggcacctccgtgacagtgtcctctaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgacagccaggggatttcaccactcaaaggccagacctgcagacgcccagattatgagacacactcgag

SEQ ID NO: 11 - protein, 21 aa, Artificial Sequence
  synthetic polypeptide
MALPVTALLLPLALLLHAARP

SEQ ID NO: 12 - protein, 15 aa, Artificial Sequence
  synthetic polypeptide
GGGGSGGGGSGGGGS

SEQ ID NO: 13 - protein, 26 aa, Artificial Sequence
  synthetic polypeptide
GGGGSGGGGSGGGGSGGGGSAEKDEL

SEQ ID NO: 14 - protein, 17 aa, Artificial Sequence
  synthetic polypeptide
LYKYKSRRSFIEEKKMP

SEQ ID NO: 15 - protein, 67 aa, Artificial Sequence
  synthetic polypeptide
TTTPAPRPPTPAPTIASQPLSLRPEACRPAAGGAVHTRGLDFACDIYIWAPLAGTCGVLLLSLITLY

SEQ ID NO: 16 - protein, 42 aa, Artificial Sequence
  synthetic polypeptide
KRGRKKLLYIFKQPFMRPVQTTQEEDGCSCRFPEEEEGGCEL

SEQ ID NO: 17 - protein, 112 aa, Artificial Sequence
  synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 120 aa, Artificial Sequence
  synthetic polypeptide
QVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSS

SEQ ID NO: 19 - protein, 107 aa, Artificial Sequence
  synthetic polypeptide
IVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRR

SEQ ID NO: 20 - protein, 116 aa, Artificial Sequence
  synthetic polypeptide
QLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSS

SEQ ID NO: 21 - protein, 113 aa, Artificial Sequence
  synthetic polypeptide
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKR

SEQ ID NO: 22 - protein, 242 aa, Artificial Sequence
  synthetic polypeptide
IVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRGGGGSGGGGSGGGGSQVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSS

SEQ ID NO: 23 - protein, 244 aa, Artificial Sequence
  synthetic polypeptide
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRGGGGSGGGGSGGGGSQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSS

SEQ ID NO: 24 - protein, 4 aa, Artificial Sequence
  synthetic polypeptide
KDEL

SEQ ID NO: 25 - protein, 4 aa, Artificial Sequence
  synthetic polypeptide
KKDE

SEQ ID NO: 26 - protein, 4 aa, Artificial Sequence
  synthetic polypeptide
  misc_feature (3)..(4): Xaa can be any naturally occurring amino acid
KKXX

SEQ ID NO: 27 - protein, 4 aa, Artificial Sequence
  synthetic polypeptide
KKMP

SEQ ID NO: 28 - protein, 4 aa, Artificial Sequence
  synthetic polypeptide
YQRL

SEQ ID NO: 29 - protein, 6 aa, Artificial Sequence
  synthetic polypeptide
AEKDEL

SEQ ID NO: 30 - protein, 26 aa, Artificial Sequence
  synthetic polypeptide
GGGGSGGGGSGGGGSGGGGSAEKDEL

SEQ ID NO: 31 - protein, 17 aa, Artificial Sequence
  synthetic polypeptide
LYKYKSRRSFIEEKKMP

SEQ ID NO: 32 - protein, 4 aa, Artificial Sequence
  synthetic polypeptide
  MOD_RES (1)..(4): wherein n is 1-10
GGGS

SEQ ID NO: 33 - protein, 4 aa, Artificial Sequence
  synthetic polypeptide
  MOD_RES (1)..(4): wherein n is 1-10
GGSG

SEQ ID NO: 34 - protein, 5 aa, Artificial Sequence
  synthetic polypeptide
  MOD_RES (1)..(5): wherein n is 1-10
GGSGG

SEQ ID NO: 35 - protein, 5 aa, Artificial Sequence
  synthetic polypeptide
  MOD_RES (1)..(5): wherein n is 1-10
GGGGS

SEQ ID NO: 36 - protein, 5 aa, Artificial Sequence
  synthetic polypeptide
GGGGS

SEQ ID NO: 37 - protein, 5 aa, Artificial Sequence
  synthetic polypeptide
GGGGS

SEQ ID NO: 38 - protein, 10 aa, Artificial Sequence
  synthetic polypeptide
GGGGSGGGGS

SEQ ID NO: 39 - protein, 20 aa, Artificial Sequence
  synthetic polypeptide
GGGGSGGGGSGGGGSGGGGS

SEQ ID NO: 40 - protein, 4 aa, Artificial Sequence
  synthetic polypeptide
YGRL

SEQ ID NO: 41 - protein, 4 aa, Artificial Sequence
  synthetic polypeptide
YQRL

SEQ ID NO: 42 - protein, 4 aa, Artificial Sequence
  synthetic polypeptide
YKGL

SEQ ID NO: 43 - protein, 4 aa, Artificial Sequence
  synthetic polypeptide
  misc_feature (2)..(3): Xaa can be any naturally occurring amino acid
YXXL

SEQ ID NO: 44 - DNA, 20 bp, Artificial Sequence
  synthetic polynucleotide
caaagagattacgaatgcct

SEQ ID NO: 45 - DNA, 20 bp, Artificial Sequence
  synthetic polynucleotide
cttgtagatatcctgatcat

SEQ ID NO: 46 - DNA, 20 bp, Artificial Sequence
  synthetic polynucleotide
cacgcacctggacagctgac

SEQ ID NO: 47 - DNA, 20 bp, Artificial Sequence
  synthetic polynucleotide
ggttgtgttgatacaagtcc

SEQ ID NO: 48 - DNA, 20 bp, Artificial Sequence
  synthetic polynucleotide
agcttggaacagactcacgg

SEQ ID NO: 49 - DNA, 20 bp, Artificial Sequence
  synthetic polynucleotide
aatgggcggaggagagtagt

SEQ ID NO: 50 - DNA, 21 bp, Artificial Sequence
  synthetic polynucleotide
cctttccgggactttcgcttt

SEQ ID NO: 51 - DNA, 20 bp, Artificial Sequence
  synthetic polynucleotide
gcagaatccaggtggcaaca

SEQ ID NO: 52 - DNA, 20 bp, Artificial Sequence
  synthetic polynucleotide
agggaactgtgggatgactg

SEQ ID NO: 53 - DNA, 20 bp, Artificial Sequence
  synthetic polynucleotide
gaacacctgagcctttgagc

(19) United States
(12) Patent Application Publication
(10) Pub. No.: US 2021/0214439 A1
(43) Pub. Date: Jul. 15, 2021

(54) CHIMERIC ANTIGEN RECEPTORS AND GENE EDITING OF CD2 FOR IMMUNOTHERAPY OF T-CELL MALIGNANCIES

(71) Applicant: National University of Singapore, Singapore (SG)

(72) Inventor: Dario Campana, Singapore (SG)

(21) Appl. No.: 17/057,978

(22) PCT Filed: May 23, 2019
(86) PCT No.: PCT/US2019/033837

§ 371 Date: Nov. 23, 2020

Related U.S. Application Data
(60) Provisional application No. 62/675,525, filed on May 23, 2018.

(51) Int. Cl. C07K 16/28; C07K 14/705; C07K 14/725; A61K 35/17

(52) U.S. Cl. CPC C07K 16/2806; C07K 14/70517; C07K 14/70578; C07K 14/7051; C07K 2317/622; C07K 2319/03; C07K 2319/02; C07K 2319/33; C12N 2310/20; A61K 35/17

(57) ABSTRACT

The present invention provides gene edited or gene silenced immune cells comprising an chimeric antigen receptor (CAR) such as an anti-CD2 CAR. In some embodiments, such engineered immune cells lack CD2 expression. Also, provided herein are methods of using such cells in cancer therapies.

### CROSS-REFERENCE TO RELATED APPLICATIONS

This application is a United States National Stage Application filed under 35 U.S.C. § 371 of International Patent Application No. PCT/US2019/33837 filed May 23, 2019 and published as WO 2019/226946 A1, which claims priority to U.S. Provisional Application No. 62/675,525 filed May 23, 2018, the entire contents of which are incorporated herein by reference in its entirety.

### REFERENCE TO A SEQUENCE LISTING

The instant application contains a Sequence Listing which has been submitted electronically in ASCII format and is hereby incorporated by reference in its entirety. Said ASCII copy, created on May 23, 2019, is named “119419_5005_WO_SequenceListing_ST25.txt” and is 44.0 kilobytes in size.

### FIELD OF THE INVENTION

The invention described herein relates generally to a clinically effective population of chimeric antigen receptor T cells (CAR-T cells) lacking CD2 expression by way of a CD2 genetic modification. The invention also relates to the use of such CAR-T cells to treat T cell malignancies.

### BACKGROUND OF THE INVENTION

Genetically-engineered immune cells are a powerful new treatment for cancer and autoimmune diseases. Results of recent clinical trials with T lymphocytes expressing chimeric antigen receptors (CARs) have provided compelling demonstration of the power of this approach. Chimeric antigen receptors (CARs) can redirect immune cells to specifically recognize and kill tumor cells. CARs are artificial multi-molecular proteins constituted by a single-chain variable region (scFv) of an antibody linked to a signaling molecule via a transmembrane domain. When the scFv ligates its cognate antigen, signal transduction is triggered, resulting in tumor cell killing by CAR-expressing cytotoxic T lymphocytes (Eshhar Z, Waks T, et al. PNAS USA. 90(2):720-724, 1993; Geiger T L, et al. J Immunol. 162(10):5931-5939, 1999; Brentjens R J, et al. Nat Med. 9(3):279-286, 2003; Cooper L J, et al. Blood 101(4):1637-1644, 2003; Imai C, et al. Leukemia. 18:676-684, 2004). Clinical trials with CAR-expressing autologous T lymphocytes have shown positive responses in patients with B-cell refractory leukemia and lymphoma (see, e.g., Till B G, et al. Blood 119(17):3940-3950, 2012; Maude S L, et al. N Engl J Med. 371(16):1507-1517, 2014).

It has been shown that CAR-T cells specific for the surface molecule CD19 induced morphologic and molecular remissions in patients with treatment-refractory CD19-positive malignancies, such as acute lymphoblastic leukemia (ALL), chronic lymphocytic leukemia, and non-Hodgkin lymphoma. Other malignancies can be attacked by T cells redirected against different antigens. Hence, the possible applications for genetically-engineered cellular therapy in oncology are wide-ranging.

The initial clinical experience with CAR-T cell infusions has also identified potential limitations, which could seriously diminish therapeutic effect and hamper development. A major issue is the variable fitness of immune cells collected from patients with cancer, resulting in an unpredictable capacity to expand in vivo, and exert anti-tumor effects. This variability complicates the identification of the most effective cell dosages, might lead to the infusion of short-lived and ineffective cell products, and could ultimately prevent the development of a consistent “living drug”. The use of T lymphocytes from healthy donors should improve effectiveness and consistency, but carries the risk of graft-versus-host disease (GvHD), a serious, and potentially fatal, consequence of donor lymphocyte infusion. In such allogeneic setting, additional modifications to the infused T cells are required to suppress their capacity to recognize tissue antigens expressed by indispensable cells.

The advent of practical methodologies for gene editing has opened new opportunities for therapeutic cell engineering which are applicable to cell therapy of cancer. Zinc finger nucleases (ZFNs), transcription activator-like effector nucleases (TALENs), and Clustered Regularly Interspaced Short Palindromic Repeats (CRISPR)/CRISPR associated (Cas) systems can be used to delete the genes encoding TCRαβ chains leading to T cells that lack alloreactivity, while other genes can be targeted to delay rejection. A report using TALEN deletion of the TCRα and CD52 loci together with anti-CD19 CAR expression indicates that combining CAR-expression with gene editing is feasible in a clinical setting, although technically challenging.

In sum, there is a significant unmet need for new therapeutic options for patients with cancer and autoimmune diseases.

### SUMMARY OF THE INVENTION

In one aspect, provided herein is an engineered immune cell comprising a CD2 blocking polypeptide, a genetic modification of a target CD2 nucleic acid sequence, and a chimeric antigen receptor (CAR) that binds CD2.

The invention describes an engineered immune cell comprising:

(i) a chimeric antigen receptor (CAR) comprising a CD2 targeting domain, a transmembrane domain, and an intracellular signaling domain; and

(ii) a genetic modification of a target CD2 nucleic acid sequence to downregulate endogenous CD2 expression in said cell.

In some embodiments, the genetic modification of the engineered immune cell comprises genome editing using a system selected from the group consisting of CRISPR/Cas, zinc finger nucleases, TALENs, and meganucleases. In some instances, the CRISPR/Cas system comprises a guide RNA corresponding to a nucleic acid sequence selected from the group consisting of SEQ ID NOS:44-47.

In some embodiments, the genetic modification comprises gene silencing using a system selected from the group consisting of short hairpin RNA, double-stranded RNA, sdRNA, short inhibitory RNA, and microRNA.

In some embodiments, the CD2 targeting domain comprises a scFv comprising a variable heavy chain (V_{H}) sequence having at least 90% sequence identity to SEQ ID NO:18 and a variable light chain (V_{L}) sequence having at least 90% sequence identity to SEQ ID NO:19. In some embodiments, the CD2 targeting domain comprises a scFv comprising variable heavy chain (V_{H}) sequence having at least 90% sequence identity to SEQ ID NO:20 and a variable light chain (V_{L}) sequence having at least 90% sequence identity to SEQ ID NO:21.

In some embodiments, the transmembrane domain of the CAR comprises a hinge-transmembrane domain of CD8α.

In some embodiments, the intracellular signaling domain of the CAR comprises one or more selected from the group consisting of a 4-1BB signaling domain, a CD28 signaling domain, an OX40 signaling domain, and a CD3ζ signaling domain.

In some embodiments, the CAR is an anti-CD2-4-1BB-CD3ζ CAR comprising an amino acid sequence having at least 90% sequence identity to SEQ ID NO:5.

In some embodiments, the engineered immune cell proliferates at a substantially equivalent rate as a comparable immune cell.

In some embodiments, the engineered immune cell proliferates at a substantially equivalent rate as a comparable immune cell. In some embodiments, the engineered immune cell is an allogeneic cell. In certain embodiments, the engineered immune cell is an autologous cell. In some embodiments, the engineered immune cell is an engineered T cell. In particular embodiments, the engineered immune cell is an engineered NK cell.

In some embodiments, any of the engineered immune cells described herein further comprise a blocking polypeptide comprising a single chain variable fragment (scFv) linked to the N-terminus of a cellular localizing domain, wherein said scFv binds to a cell surface molecule, wherein said cellular localizing domain comprises an amino acid sequence selected from the group consisting of an endoplasmic reticulum (ER) retention sequence, a Golgi retention sequence, and a proteosome localizing sequence, and wherein said blocking polypeptide remains intracellularly within said engineered cell and binds the endogenous cell surface molecule within the engineered cell.

In some embodiments, the ER retention sequence of the blocking polypeptide comprises an amino acid sequence selected from the group consisting of KDEL (SEQ ID NO:24), KKXX (SEQ ID NO:26), and KKMP (SEQ ID NO:27), wherein X is any amino acid; or said Golgi retention sequence is selected from the group consisting of YGRL (SEQ ID NO:40), YQRL (SEQ ID NO:41), YKGL (SEQ ID NO:42), and YXXL (SEQ ID NO:43), wherein X is any amino acid.

In some embodiments, the blocking polypeptide further comprises a transmembrane domain linked between said scFv and either said ER retention sequence domain comprising KKMP or KKTN or said Golgi retention sequence domain comprising YGRL, YQRL, YKGL, wherein said transmembrane domain is a transmembrane domain selected from any one of the group consisting of CD8α, CD8β, 4-IBB, CD28, CD34, CD4, FcεRIγ, CD16, OX40, CD3ζ, CD3ε, CD3γ, CD3δ, TCRα, CD32, CD64, VEGFR2, FAS, and FGFR2B. In some embodiments, the transmembrane domain comprises a hinge-transmembrane domain of CD8α.

In another aspect, the invention provides a pharmaceutical composition comprising at least one of the engineered immune cells described herein. In some embodiments, the pharmaceutical composition comprises an isolated population of any one of the engineered immune cells outlined herein.

In another aspect, the invention provides a method of treating cancer in a subject in need thereof comprising administering a therapeutically effective amount of a composition comprising any one of the engineered immune cells described herein to the subject, thereby treating cancer in a subject in need thereof. In some embodiments, the method includes administering a therapeutic composition of a population of the engineered immune cells described herein.

In some embodiments, the composition also includes a pharmaceutically acceptable carrier. In other word, provided herein is a pharmaceutical composition comprising at least one of the engineered immune cells described herein. In some cases, the pharmaceutical composition also includes a pharmaceutically acceptable carrier.

In some embodiments, the cancer is a T-cell malignancy or a CD2 associated cancer. In some embodiments, the T-cell malignancy or the CD2 associated cancer is selected from the group consisting of T cell leukemia T cell lymphoma, T-cell acute lymphoblastic leukemia (T-ALL), early T-cell progenitor acute lymphoblastic leukemia (ETP-ALL), T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, cutaneous T-cell lymphomas (CTCL), any subtype of CTCL, mycosis fungoides, Sézary syndrome, primary cutaneous gamma-delta T-cell lymphoma, a malignancy with the T lineage subsets of Non-Hodgkin's lymphoma (NHL), peripheral T-cell lymphoma (PTCL), PTCL not otherwise specified (PTCL-NOS), angioimmunoblastic T-cell lymphoma, and anaplastic large cell lymphoma.

In some embodiments, the administration is by any one selected from the group consisting of intravenous infusion, intra-arterial infusion, intraperitoneal infusion, direct injection into tumor and/or perfusion of tumor bed after surgery, implantation at a tumor site in an artificial scaffold, or intrathecal administration.

Provided herein is an expression vector composition comprising an expression vector comprising a polynucleotide encoding any one of the CARs outlined herein.

Provided herein is also an expression vector comprising a polynucleotide encoding a CRISPR guide RNA complementary to the human CD2 gene, wherein the polynucleotide corresponds to a nucleic acid sequence selected from the group consisting of SEQ ID NOS:44-47.

In some embodiments, provided herein is an expression vector composition comprising an expression vector comprising the polynucleotide encoding any one of the blocking polypeptides outlined herein.

In one aspect, the present invention describes a method for producing an engineered immune cell of any one of the embodiments. Such method comprises: introducing an expression vector (or expression vector composition) comprising a polynucleotide encoding a CAR and an expression vector (or expression vector composition) comprising one or more polynucleotides encoding one or more CRISPR guide RNAs into an immune cell. In some embodiments, the method also includes introducing an expression vector (or expression vector composition) comprising the polynucleotide encoding a blocking polypeptide. In some embodiments, introducing comprises concomitantly or sequentially introducing the expression vectors or expression vector compositions into said immune cell

The invention provides is a simple and effective method for the blockade of surface receptor expression. Specific constructs, named Protein Expression Blockers (PEBLs), prevent transport of targeted proteins to the cell membrane. PEBL constructs can be readily combined with genetic modification constructs and be incorporated into existing large-scale cGMP-grade protocols for ex vivo cell processing to optimize the function of immune cells.

### BRIEF DESCRIPTION OF THE DRAWINGS

FIG. 1 depicts a schematic of an exemplary anti-CD2 chimeric antigen receptor (CAR) construct described herein.

FIG. 2 illustrates expression of an anti-CD2 CAR in Jurkat cells. The anti-CD2 CAR comprise an anti-CD2 scFv based on the anti-CD2 monoclonal antibody 9.6. Detailed descriptions of 9.6 can be found, e.g., in Kamoun et al. J Exp Med, 1981, 153:207-212. Cells were transduced with vectors containing the CAR construct and GFP or GFP only (“Mock”). Flow cytometric dot plots illustrate anti-CD2 CAR expression. Anti-goat anti-mouse antibody APC (GAM-APC) was used.

Detailed descriptions of 9.6 and 9-1 anti-CD2 monoclonal antibodies can be found, e.g., in Kamoun et al. J Exp Med, 1981, 153:207-212 and in Bernard et al., in Leukocyte Typing II, 1986, eds. Reinherz, E. L., Haynes, B. F., Nadler, L. M., & Bernstein, I.D. (Springer, New York), pp. 53-66, respectively.

FIG. 3 shows that expression of an anti-CD2 CAR induced expression of activation markers in the presence of CD2+ target cells. The bar graphs show an increased number of CD25+ cells and CD69+ cells of the CCRF-CEM cell line when in the presence of cells expressing the 9.6 anti-CD2 CAR.

FIG. 4 shows expression of an 9.6 anti-CD2 CAR construct on T cells. T cells were transduced with vectors containing the CAR construct and GFP or GFP only (“Mock”). Flow cytometric dot plots illustrate anti-CD2 CAR expression. An anti-goat anti-mouse antibody APC (GAM-APC) was used.

FIG. 5 shows cytoxicity activity of 9.6 anti-CD2 CAR expressing T cells against target cells (CD2+ target cells). Cytotoxicity of CAR- or mock-transduced T cells electroporated either with anti-CD2 scFv-41BB-CD3ζ CAR mRNA or GFP only mRNA was shown in a coculture experiment. CAR T cells and target were plated at a 1:1 effector-to-target ratio (E:T). After several days of co-culture, the number of viable target cells was determined. The bar graph shows that 9.6 anti-CD2 CAR T cells exerted cytotoxicity on the CD2+ target cells. CD107a represents a marker for CD8+ T cell degranulation following stimulation and NK cell functional activity. The bar graph shows that a higher percentage of CD107a+ cells when expressing the 9.6 anti-CD2 CAR compared to GFP only.

FIG. 6 provides exemplary embodiments of anti-CD2 protein expression blocker (PEBL) constructs described herein. The 9.6 PEBL I construct includes a CD8α signal peptide, a 9.6 anti-CD2 scFv comprising the VL domain connected to the VH domain via a linker, and an ER retention domain. The 9.6 PEBL II construct includes a CD8α signal peptide, a 9.6 anti-CD2 scFv comprising the VL domain connected to the VH domain via a linker, a CD8α hinge-transmembrane domain, and an ER retention domain. The 9-1 PEBL I construct includes a CD8α signal peptide, a 9-1 anti-CD2 scFv comprising the VL domain connected to the VH domain via a linker, and an ER retention domain. The 9-1 PEBL II construct includes a CD8α signal peptide, a 9-1 anti-CD2 scFv comprising the VL domain connected to the VH domain via a linker, a CD8α hinge-transmembrane domain, and an ER retention domain.

Detailed descriptions of 9.6 anti-CD2 monoclonal antibody and 9-1 anti-CD2 monoclonal antibody can be found, e.g., in Kamoun et al. J Exp Med, 1981, 153:207-212 and in Bernard et al., in Leukocyte Typing II, 1986, eds. Reinherz, E. L., Haynes, B. F., Nadler, L. M., & Bernstein, I.D. (Springer, New York), pp. 53-66, respectively. The 9.6 scFv recognizes and binds CD2 on both resting and activated T cells. It also inhibits (blocks) binding of CD58 to CD2. The 9-1 scFv recognizes and binds CD2 on activated T cells. It does not block CD58 binding to CD2.

FIG. 7 shows flow cytometry histograms of surface and intracellular expression of CD2 in Jurkat cells transduced with a 9.6 anti-CD2 PEBL I, a 9.6 anti-CD2 PEBL II, a 9-1 anti-CD2 PEBL I, a 9-1 anti-CD2 PEBL II, or GFP alone (“Mock”). Expression of the 9.6 anti-CD2 PEBL II construct in Jurkat cells downregulated expression of CD2.

FIG. 8 shows flow cytometry dot plots of surface expression of CD2 in T cells electroporated with 9.6 anti-CD2 PEBL II construct. The data shows downregulation (partial downregulation) of CD2 expression by electroporated T cells.

FIG. 9 shows flow cytometry histograms of surface and intracellular expression of CD2 in engineered Jurkat cells that have undergone CRISPR/Cas9 based genome editing of the CD2 locus and express the 9.6 anti-CD2 PEBL II construct.

FIG. 10 shows CD2 cleavage products in Jurkat cells that have undergone CRISPR/Cas9 based genome editing of the CD2 locus.

FIG. 11A, FIG. 11B and FIG. 11C show expression of CD2 on transduced Jurkat cells as determined by flow cytometry. Jurkat cells were transduced with the indicated lentiviruses at MOI 1 or 5 and analysed by flow cytometry for CD2 expression 8 days after transduction.

### DETAILED DESCRIPTION OF THE INVENTION

A description of example embodiments of the invention follows.

### I. Introduction

The present invention provides a method that allows rapid and efficient downregulation of surface molecules in T cells, including CAR-T cells. In one embodiment of the present invention, provided are CD2ΔCD2CAR-T cells that are CD2 specific CAR-T cells that do not express CD2 via genetic modification (e.g., gene editing or gene silencing). In some embodiments, the CD2ΔCD2CAR-T cells also express a PEBL construct outlined herein that blocks expression of another cell surface molecule or a secreted molecule such as, but not limited to a cytokine. CD2 blockade via genetic modification is durable and does not affect expression of other surface molecules. CD2ΔCD2CAR-T cells can survive and proliferate as well as comparable T cells including CD2 CAR-T cells.

### II. Definitions

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

As used herein, an “engineered immune cell” refers to an immune cell that has been genetically modified as compared to a naturally-occurring immune cell.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. In the case of a double-stranded polymer, “nucleic acid” can refer to either or both strands of the molecule.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In certain embodiments, the nucleotide sequence encoding, e.g., a target-binding molecule linked to a localizing domain is a heterologous sequence (e.g., a gene that is of a different species or cell type origin).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

As will be appreciated by those of skill in the art, in some aspects, the nucleic acid further comprises a plasmid sequence. The plasmid sequence can include, for example, one or more sequences selected from the group consisting of a promoter sequence, a selection marker sequence, and a locus-targeting sequence.

As used herein, the gene encoding a target-binding molecule linked to a localizing domain is sometimes referred to as “gene encoding a PEBL,” “polynucleotide encoding a PEBL,” “nucleic acid encoding a PEBL,” and the like.

In certain embodiments, the target-binding molecule is an antibody or antigen-binding fragment thereof. As used herein, “antibody” means an intact antibody or antigen-binding fragment of an antibody, including an intact antibody or antigen-binding fragment that has been modified or engineered, or that is a human antibody. Examples of antibodies that have been modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)2, Fv, single chain antibodies (e.g., scFv), minibodies and diabodies.

A “diabody” is a small antibody fragment with two antigen-binding sites. The fragments comprise a heavy chain variable region (V_{H}) connected to a light chain variable region (V_{L}) in the same polypeptide chain (V_{H}-VL or V_{L}-V_{H}). By using a linker that is too short to allow pairing between the two domains on the same chain, the domains are forced to pair with the complementary domains of another chain and create two antigen-binding sites. Diabodies are described in, e.g., patent documents EP 404,097; WO 93/11161; and Holliger et al, (1993) Proc. Natl. Acad. Sci. USA 90: 6444-6448.

In certain embodiments, the antibody is a triabody or a tetrabody. Methods of designing and producing triabodies and tetrabodies are known in the art. See, e.g., Todorovska et al, J. Immunol. Methods 248(1-2):47-66, 2001.

A “domain antibody fragment” is an immunologically functional immunoglobulin fragment containing only the variable region of a heavy chain or the variable region of a light chain. In some instances, two or more VH regions are covalently joined with a peptide linker to create a bivalent domain antibody fragment. The two VH regions of a bivalent domain antibody fragment may target the same or different antigens.

In some embodiments, the antibody is modified or engineered. Examples of modified or engineered antibodies include chimeric antibodies, multiparatopic antibodies (e.g., biparatopic antibodies), and multispecific antibodies (e.g., bispecific antibodies).

As used herein, “multiparatopic antibody” means an antibody that comprises at least two single domain antibodies, in which at least one single domain antibody is directed against a first antigenic determinant on an antigen and at least one other single domain antibody is directed against a second antigenic determinant on the same antigen. Thus, for example, a “biparatopic” antibody comprises at least one single domain antibody directed against a first antigenic determinant on an antigen and at least one further single domain antibody directed against a second antigenic determinant on the same antigen.

As used herein, “multispecific antibody” means an antibody that comprises at least two single domain antibodies, in which at least one single domain antibody is directed against a first antigen and at least one other single domain antibody is directed against a second antigen (different from the first antigen). Thus, for example, a “bispecific” antibody is one that comprises at least one single domain antibody directed against a first antigen and at least one further single domain antibody directed against a second antigen, e.g., different from the first antigen.

In some embodiments, the antibodies disclosed herein are monoclonal antibodies, e.g., murine monoclonal antibodies. Methods of producing monoclonal antibodies are known in the art. See, for example, Pluckthun (1994) The Pharmacology of Monoclonal Antibodies, Vol. 113, Rosenburg and Moore eds. Springer-Verlag, New York, pp. 269-315.

A “Fab fragment” comprises one light chain and the CH1 and variable regions of one heavy chain. The heavy chain of a Fab molecule cannot form a disulfide bond with another heavy chain molecule.

An “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion of one heavy chain that contains the VH domain and the CHI domain and also the region between the CHI and CH2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}² domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

In a particular embodiment, the target-binding molecule is single-chain Fv antibody (“scFv antibody”). scFv refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun (1994) The Pharmacology Of Monoclonal Antibodies, vol. 113, Rosenburg and Moore eds. Springer-Verlag, New York, pp. 269-315. See also, PCT Publication No. WO 88/01649 and U.S. Pat. Nos. 4,946,778 and 5,260,203.

The term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 90% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et ah, Current Protocols in Molecular Biology). One example of algorithm that is suitable for determining percent sequence identity and sequence similarity is the BLAST algorithm, which is described in Altschul et al, J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

As used herein, “operatively linked” in the context of a PEBL gene refers to a gene encoding a target-binding molecule directly in frame (e.g., without a linker) adjacent to one or more genes encoding one or more localizing domains. Alternatively, the gene encoding a target-binding molecule may be connected to one or more gene encoding one or more localizing domains through a linker sequence, as described herein.

As used herein, “linked” in the context of a protein refers to the joining of a first domain, e.g., a target-binding molecule to a second domain, e.g., a localizing domain. The linker can be an amino acid sequence. Various suitable linkers known in the art can be used to tether the target-binding molecule to a localizing domain. For example, non-naturally occurring peptides, such as a polypeptide consisting of hydrophilic residues of varying length, or a (GGGGS)ₙ (SEQ ID NO:35) polypeptide, in which n is an integer of, e.g., 3-12, inclusive, can be used according to the present invention.

As used herein, the terms “treat,” “treating,” or “treatment,” refer to counteracting a medical condition (e.g., a condition related to a T cell malignancy) to the extent that the medical condition is improved according to a clinically-acceptable standard.

As used herein, “subject” refers to a mammal (e.g., human, non-human primate, cow, sheep, goat, horse, dog, cat, rabbit, guinea pig, rat, mouse). In certain embodiments, the subject is a human. A “subject in need thereof” refers to a subject (e.g., patient) who has, or is at risk for developing, a disease or condition that can be treated (e.g., improved, ameliorated, prevented) by inducing T cells to exert specific cytotoxicity against malignant T cells.

As defined herein, a “therapeutically effective amount” refers to an amount that, when administered to a subject, is sufficient to achieve a desired therapeutic effect (treats a condition related to a T cell malignancy) in the subject under the conditions of administration. An effective amount of the agent to be administered can be determined by a clinician of ordinary skill using the guidance provided herein and other methods known in the art, and is dependent on several factors including, for example, the particular agent chosen, the subject's age, sensitivity, tolerance to drugs and overall well-being.

As used herein, “enhanced therapeutic efficacy” refers to one or more of reduced graft-versus-host disease (GvHD) in a host, reduced or elimination of rejection by a host, extended survival in a host, reduced inhibition by the tumor in a host, reduced self-killing in a host, reduced inflammatory cascade in a host, or sustained CAR-mediated signal transduction in a host.

### III. Protein Expression Blockers

The methods described herein enable rapid removal or inactivation of specific target proteins such as cell surface molecules or secreted molecules. The method relies, in part, on a polypeptide construct containing a target-binding molecule that binds a target (e.g., protein) to be removed, neutralized or blocked. The target-binding molecule is linked to a domain (e.g., a localizing domain or intracellular retention domain) that directs the polypeptide to specific cellular compartments, such as the Golgi, endoplasmic reticulum, proteasome, or cellular membrane, depending on the application. For simplicity, a target-binding molecule linked to a localizing domain is sometimes referred to herein as “Protein Expression Blocker” or “PEBL”. In some embodiments, the PEBL also include a signal peptide domain. In yet other embodiments, the PEBL contains a transmembrane domain or the cellular localizing domain includes a transmembrane domain.

In some embodiments, the PEBL binds a target molecule selected from the group consisting of a cell surface molecule or a secreted molecule. In some cases, the target molecule is a cytokine such as but not limited to IL-6, IL-2, IL-4, IL-7, IL-10, IL-12, IL-15, IL-18, IL-21, IL-27, IL-35, interferon (IFN)-γ, IFN-β, IFN-α, tumor necrosis factor (TNF)-α, and transforming growth factor (TGF)-β. In some cases, the target molecule is a CD protein (CD antigen) expressed on an immune cell such as a T cell. In some cases, the CD protein is selected from the group consisting of CD1b, CD1c, CD1d, CD3 d, CD3e, CD4, CD7, CD8, CD11, CD13, CD16b, CD25, CD26, CD27, CD28, CD30, CD33, CD34, CD38, CD39, CD40, CD44, CD45, CD48, CD56, CD58, CD59, CD62L, CD66a, CD69, CD70, CD80, CD83, CD85, CD86, CD86, CD95, CD96, CD99, CD100, CD102, CD103, CD107a, CD107b, CD111, CD112, CD117, CD119, CD122, CD126, CD127, CD132, CD134, CD137, CD150, CD152, CD153, CD154, CD155, CD158, CD160, CD161, CD178, CD183, CD185, CD191, CD192, CD196, CD212, CD215, CD218, CD223, CD226, CD244, CD252, CD253, CD275, CD278, CD279, CD281, CD282, CD283, CD284, CD286, CD305, CD314, CD336, CD337, CD355, and CD360.

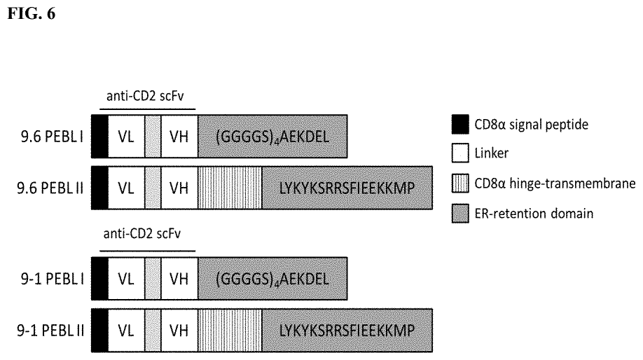

Non-limiting examples of PEBLs are shown in FIG. 6 and exemplary amino acid and nucleic acid sequences are provided in Table 1.

TABLE 1
Sequences of PEBLs, CARs and components thereof

| Name | SEQ ID NO: | Sequence |
|---|---|---|
| 9-1 PEBL I | SEQ ID NO: 1 | MALPVTALLLPLALLLHAARPIVMTQSPATLSVTPGDRVSLSCRASQSIS |
| | | DYLHWYQQKSHESPRLLIKYASQSISGIPSRFSGSGSGSDFTLSINSVEP |
| | | EDVGVYYCQNGHSFPLTFGAGTKLELRRGGGGSGGGGSGGGGSQVQLQQP |
| | | GTELVRPGSSVKLSCKASGYTFTSYWVNWVKQRPDQGLEWIGRIDPYDSE |
| | | THYNQKFTDKAISTIDTSSNTAYMQLSTLTSDASAVYYCSRSPRDSSTNL |
| | | ADWGQGTLVTVSSGGGGSGGGGSGGGGSGGGGSAEKDEL |
| 9-1 PEBL II | SEQ ID NO: 2 | MALPVTALLLPLALLLHAARPIVMTQSPATLSVTPGDRVSLSCRASQSIS |
| | | DYLHWYQQKSHESPRLLIKYASQSISGIPSRFSGSGSGSDFTLSINSVEP |
| | | EDVGVYYCQNGHSFPLTFGAGTKLELRRGGGGSGGGGSGGGGSQVQLQQP |
| | | GTELVRPGSSVKLSCKASGYTFTSYWVNWVKQRPDQGLEWIGRIDPYDSE |
| | | THYNQKFTDKAISTIDTSSNTAYMQLSTLTSDASAVYYCSRSPRDSSTNL |
| | | ADWGQGTLVTVSSKPTTTPAPRPPTPAPTIASQPLSLRPEACRPAAGGAV |
| | | HTRGLDFACDIYIWAPLAGTCGVLLLSLVITLYKYKSRRSFIEEKKMP |
| 9.6 PEBL I | SEQ ID NO: 3 | MALPVTALLLPLALLLHAARPNIMMTQSPSSLAVSAGEKVTMTCKSSQSV |
| | | LYSSNQKNYLAWYQQKPGQSPKLLIYWASTRESGVPDRFTGSGSGTDFTL |
| | | TISSVQPEDLAVYYCHQYLSSHTFGGGTKLEIKRGGGGSGGGGSGGGGSQ |
| | | LQQPGAELVRPGSSVKLSCKASGYTFTRYWIHWVKQRPIQGLEWIGNIDP |
| | | SDSETHYNQKFKDKATLTVDKSSGTAYMQLSSLTSEDSAVYYCATEDLYY |
| | | AMEYWGQGTSVTVSSGGGGSGGGGSGGGGSGGGGSAEKDEL |
| 9.6 PEBL II | SEQ ID NO: 4 | MALPVTALLLPLALLLHAARPNIMMTQSPSSLAVSAGEKVTMTCKSSQSV |
| | | LYSSNQKNYLAWYQQKPGQSPKLLIYWASTRESGVPDRFTGSGSGTDFTL |
| | | TISSVQPEDLAVYYCHQYLSSHTFGGGTKLEIKRGGGGSGGGGSGGGGSQ |
| | | LQQPGAELVRPGSSVKLSCKASGYTFTRYWIHWVKQRPIQGLEWIGNIDP |
| | | SDSETHYNQKFKDKATLTVDKSSGTAYMQLSSLTSEDSAVYYCATEDLYY |
| | | AMEYWGQGTSVTVSSKPTTTPAPRPPTPAPTIASQPLSLRPEACRPAAGG |
| | | AVHTRGLDFACDIYIWAPLAGTCGVLLLSLVITLYKYKSRRSFIEEKKMP |
| 9.6 anti-CD2 | SEQ ID NO: 5 | MALPVTALLLPLALLLHAARPNIMMTQSPSSLAVSAGEKVTMTCKSSQSV |
| CAR | | LYSSNQKNYLAWYQQKPGQSPKLLIYWASTRESGVPDRFTGSGSGTDFTL |
| | | TISSVQPEDLAVYYCHQYLSSHTFGGGTKLEIKRGGGGSGGGGSGGGGSQ |
| | | LQQPGAELVRPGSSVKLSCKASGYTFTRYWIHWVKQRPIQGLEWIGNIDP |
| | | SDSETHYNQKFKDKATLTVDKSSGTAYMQLSSLTSEDSAVYYCATEDLYY |
| | | AMEYWGQGTSVTVSSTTTPAPRPPTPAPTIASQPLSLRPEACRPAAGGAV |
| | | HTRGLDFACDIYIWAPLAGTCGVLLLSLVITLYCKRGRKKLLYIFKQPFM |
| | | RPVQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 9-1 PEBL I | SEQ ID NO: 6 | GAATTCGGCTTCCACCATGGCTCTGCCCGTGACCGCCCTGCTGCTGCCTC |
| | | TGGCTCTGCTGCTGCACGCTGCCCGCCCAATCGTGATGACCCAGAGCCCA |
| | | GCCACCCTGTCCGTGACACCTGGCGACCGGGTGTCTCTGAGCTGCAGAGC |
| | | CTCCCAGTCTATCAGCGATTACCTGCACTGGTATCAGCAGAAGTCCCACG |
| | | AGTCTCCCCGGCTGCTGATCAAGTACGCTAGCCAGTCTATCAGCGGCATC |
| | | CCTAGCCGGTTCTCCGGATCTGGAAGCGGATCCGACTTTACCCTGAGCAT |
| | | CAACTCCGTGGAGCCAGAGGATGTGGGCGTGTACTATTGCCAGAATGGCC |
| | | ACTCCTTCCCCCTGACCTTTGGCGCCGGCACAAAGCTGGAGCTGCGGAGA |
| | | GGCGGCGGCGGCTCTGGAGGAGGAGGAAGCGGAGGAGGAGGCTCCCAGGT |
| | | GCAGCTGCAGCAGCCAGGAACAGAGCTGGTGCGGCCCGGCAGCTCCGTGA |
| | | AGCTGTCCTGTAAGGCCTCTGGCTACACCTTCACAAGCTATTGGGTGAAC |
| | | TGGGTGAAGCAGAGGCCTGACCAGGGCCTGGAGTGGATCGGAAGGATCGA |
| | | CCCATACGATTCTGAGACACACTATAACCAGAAGTTTACAGACAAGGCCA |
| | | TCAGCACCATCGATACATCTAGCAATACCGCCTATATGCAGCTGTCCACC |
| | | CTGACATCTGATGCCAGCGCCGTGTACTATTGTTCTAGGAGCCCTCGCGA |
| | | CTCCTCTACAAATCTGGCAGATTGGGGACAGGGCACCCTGGTGACAGTGA |
| | | GCTCCGGTGGTGGCGGCAGTGGTGGCGGTGGCTCAGGCGGTGGTGGCTCC |
| | | GGTGGCGGTGGCTCTGCAGAAAAAGATGAGTTGTAACTCGAG |
| 9-1 PEBL II | SEQ ID NO: 7 | GAATTCGGCTTCCACCATGGCTCTGCCCGTGACCGCCCTGCTGCTGCCTC |
| | | TGGCTCTGCTGCTGCACGCTGCCCGCCCAATCGTGATGACCCAGAGCCCA |
| | | GCCACCCTGTCCGTGACACCTGGCGACCGGGTGTCTCTGAGCTGCAGAGC |
| | | CTCCCAGTCTATCAGCGATTACCTGCACTGGTATCAGCAGAAGTCCCACG |
| | | AGTCTCCCCGGCTGCTGATCAAGTACGCTAGCCAGTCTATCAGCGGCATC |
| | | CCTAGCCGGTTCTCCGGATCTGGAAGCGGATCCGACTTTACCCTGAGCAT |
| | | CAACTCCGTGGAGCCAGAGGATGTGGGCGTGTACTATTGCCAGAATGGCC |
| | | ACTCCTTCCCCCTGACCTTTGGCGCCGGCACAAAGCTGGAGCTGCGGAGA |
| | | GGCGGCGGCGGCTCTGGAGGAGGAGGAAGCGGAGGAGGAGGCTCCCAGGT |
| | | GCAGCTGCAGCAGCCAGGAACAGAGCTGGTGCGGCCCGGCAGCTCCGTGA |
| | | AGCTGTCCTGTAAGGCCTCTGGCTACACCTTCACAAGCTATTGGGTGAAC |
| | | TGGGTGAAGCAGAGGCCTGACCAGGGCCTGGAGTGGATCGGAAGGATCGA |
| | | CCCATACGATTCTGAGACACACTATAACCAGAAGTTTACAGACAAGGCCA |
| | | TCAGCACCATCGATACATCTAGCAATACCGCCTATATGCAGCTGTCCACC |
| | | CTGACATCTGATGCCAGCGCCGTGTACTATTGTTCTAGGAGCCCTCGCGA |
| | | CTCCTCTACAAATCTGGCAGATTGGGGACAGGGCACCCTGGTGACAGTGA |
| | | GCTCCAAGCCAACCACAACCCCTGCACCAAGGCCACCTACACCAGCACCT |
| | | ACCATCGCAAGCCAGCCACTGTCCCTGAGGCCAGAGGCATGTAGGCCTGC |
| | | AGCAGGAGGCGCCGTGCACACACGCGGCCTGGACTTTGCCTGCGATATCT |
| | | ACATCTGGGCACCACTGGCAGGAACCTGTGGCGTGCTGCTGCTGAGCCTG |
| | | GTGATTACCCTGTATAAGTACAAGTCCAGACGCTCATTCATTGAGGAAAA |
| | | GAAAATGCCTTAACTCGAG |
| 9.6 PEBL I | SEQ ID NO: 8 | GAATTCGGCTTCCACCATGGCTCTGCCCGTGACCGCCCTGCTGCTGCCTC |
| | | TGGCTCTGCTGCTGCACGCTGCCCGCCCAAACATCATGATGACCCAGTCC |
| | | CCCAGCTCCCTGGCCGTGTCTGCCGGAGAGAAGGTGACCATGACATGCAA |
| | | GTCTAGCCAGTCCGTGCTGTACTCCTCTAACCAGAAGAATTACCTGGCCT |
| | | GGTATCAGCAGAAGCCCGGCCAGAGCCCTAAGCTGCTGATCTATTGGGCA |
| | | AGCACCCGGGAGTCCGGAGTGCCAGACAGATTCACCGGAAGCGGATCCGG |
| | | AACAGACTTCACCCTGACAATCAGCTCCGTGCAGCCTGAGGACCTGGCCG |
| | | TGTACTATTGCCACCAGTACCTGTCTAGCCACACCTTCGGCGGCGGCACA |
| | | AAGCTGGAGATCAAGAGGGGAGGAGGAGGATCCGGAGGAGGAGGCTCTGG |
| | | CGGCGGCGGCAGCCAGCTGCAGCAGCCAGGAGCAGAGCTGGTGAGGCCCG |
| | | GCTCCTCTGTGAAGCTGTCTTGTAAGGCCAGCGGCTACACCTTCACAAGG |
| | | TATTGGATCCACTGGGTGAAGCAGCGCCCTATCCAGGGCCTGGAGTGGAT |
| | | CGGCAACATCGACCCATCTGATAGCGAGACACACTACAATCAGAAGTTTA |
| | | AGGACAAGGCCACCCTGACAGTGGATAAGAGCTCCGGCACCGCCTATATG |
| | | CAGCTGTCTAGCCTGACATCCGAGGACTCTGCCGTGTACTATTGTGCCAC |
| | | AGAGGATCTGTACTATGCCATGGAGTACTGGGGCCAGGGCACCTCCGTGA |
| | | CAGTGTCCTCTGGTGGTGGCGGCAGTGGTGGCGGTGGCTCAGGCGGTGGT |
| | | GGCTCCGGTGGCGGTGGCTCTGCAGAAAAAGATGAGTTGTAACTCGAG |
| 9.6 PEBL II | SEQ ID NO: 9 | GAATTCGGCTTCCACCATGGCTCTGCCCGTGACCGCCCTGCTGCTGCCTC |
| | | TGGCTCTGCTGCTGCACGCTGCCCGCCCAAACATCATGATGACCCAGTCC |
| | | CCCAGCTCCCTGGCCGTGTCTGCCGGAGAGAAGGTGACCATGACATGCAA |
| | | GTCTAGCCAGTCCGTGCTGTACTCCTCTAACCAGAAGAATTACCTGGCCT |
| | | GGTATCAGCAGAAGCCCGGCCAGAGCCCTAAGCTGCTGATCTATTGGGCA |
| | | AGCACCCGGGAGTCCGGAGTGCCAGACAGATTCACCGGAAGCGGATCCGG |
| | | AACAGACTTCACCCTGACAATCAGCTCCGTGCAGCCTGAGGACCTGGCCG |
| | | TGTACTATTGCCACCAGTACCTGTCTAGCCACACCTTCGGCGGCGGCACA |
| | | AAGCTGGAGATCAAGAGGGGAGGAGGAGGATCCGGAGGAGGAGGCTCTGG |
| | | CGGCGGCGGCAGCCAGCTGCAGCAGCCAGGAGCAGAGCTGGTGAGGCCCG |
| | | GCTCCTCTGTGAAGCTGTCTTGTAAGGCCAGCGGCTACACCTTCACAAGG |
| | | TATTGGATCCACTGGGTGAAGCAGCGCCCTATCCAGGGCCTGGAGTGGAT |
| | | CGGCAACATCGACCCATCTGATAGCGAGACACACTACAATCAGAAGTTTA |
| | | AGGACAAGGCCACCCTGACAGTGGATAAGAGCTCCGGCACCGCCTATATG |
| | | CAGCTGTCTAGCCTGACATCCGAGGACTCTGCCGTGTACTATTGTGCCAC |
| | | AGAGGATCTGTACTATGCCATGGAGTACTGGGGCCAGGGCACCTCCGTGA |
| | | CAGTGTCCTCTAAGCCAACCACAACCCCTGCACCAAGGCCACCTACACCA |
| | | GCACCTACCATCGCAAGCCAGCCACTGTCCCTGAGGCCAGAGGCATGTAG |
| | | GCCTGCAGCAGGAGGCGCCGTGCACACACGCGGCCTGGACTTTGCCTGCG |
| | | ATATCTACATCTGGGCACCACTGGCAGGAACCTGTGGCGTGCTGCTGCTG |
| | | AGCCTGGTGATTACCCTGTATAAGTACAAGTCCAGACGCTCATTCATTGA |
| | | GGAAAAGAAAATGCCTTAACTCGAG |
| 9.6 anti-CD2 | SEQ ID NO: 10 | GAATTCGGCTTCCACCATGGCTCTGCCCGTGACCGCCCTGCTGCTGCCTC |
| CAR | | TGGCTCTGCTGCTGCACGCTGCCCGCCCAAACATCATGATGACCCAGTCC |
| | | CCCAGCTCCCTGGCCGTGTCTGCCGGAGAGAAGGTGACCATGACATGCAA |
| | | GTCTAGCCAGTCCGTGCTGTACTCCTCTAACCAGAAGAATTACCTGGCCT |
| | | GGTATCAGCAGAAGCCCGGCCAGAGCCCTAAGCTGCTGATCTATTGGGCA |
| | | AGCACCCGGGAGTCCGGAGTGCCAGACAGATTCACCGGAAGCGGATCCGG |
| | | AACAGACTTCACCCTGACAATCAGCTCCGTGCAGCCTGAGGACCTGGCCG |
| | | TGTACTATTGCCACCAGTACCTGTCTAGCCACACCTTCGGCGGCGGCACA |
| | | AAGCTGGAGATCAAGAGGGGAGGAGGAGGATCCGGAGGAGGAGGCTCTGG |
| | | CGGCGGCGGCAGCCAGCTGCAGCAGCCAGGAGCAGAGCTGGTGAGGCCCG |
| | | GCTCCTCTGTGAAGCTGTCTTGTAAGGCCAGCGGCTACACCTTCACAAGG |
| | | TATTGGATCCACTGGGTGAAGCAGCGCCCTATCCAGGGCCTGGAGTGGAT |
| | | CGGCAACATCGACCCATCTGATAGCGAGACACACTACAATCAGAAGTTTA |
| | | AGGACAAGGCCACCCTGACAGTGGATAAGAGCTCCGGCACCGCCTATATG |
| | | CAGCTGTCTAGCCTGACATCCGAGGACTCTGCCGTGTACTATTGTGCCAC |
| | | AGAGGATCTGTACTATGCCATGGAGTACTGGGGCCAGGGCACCTCCGTGA |
| | | CAGTGTCCTCTACCACTACACCTGCACCAAGGCCTCCCACACCCGCTCCC |
| | | ACTATCGCTTCCCAGCCACTGTCCCTGAGGCCCGAGGCCTGCAGGCCAGC |
| | | AGCTGGCGGAGCCGTGCATACTAGGGGGCTGGACTTCGCTTGCGACATCT |
| | | ACATCTGGGCCCCACTGGCAGGGACATGCGGAGTCCTGCTGCTGTCCCTG |
| | | GTCATCACACTGTACTGCAAGCGGGGGCGCAAAAAACTGCTGTATATCTT |
| | | TAAGCAGCCTTTCATGAGACCAGTGCAGACAACCCAGGAGGAAGATGGGT |
| | | GCTCATGCCGGTTTCCCGAGGAGGAGGAAGGCGGCTGCGAGCTGAGGGTG |
| | | AAGTTTTCCCGCTCAGCAGATGCTCCTGCCTACCAGCAGGGCCAGAACCA |
| | | GCTGTATAATGAGCTGAACCTGGGCAGACGCGAAGAGTATGATGTGCTGG |
| | | ACAAAAGGCGGGGAAGAGACCCCGAAATGGGAGGGAAGCCAAGGCGGAAA |
| | | AACCCCCAGGAGGGCCTGTACAATGAGCTGCAGAAGGACAAAATGGCAGA |
| | | GGCTTACAGTGAGATTGGGATGAAGGGAGAGAGACGGAGGGGAAAAGGGC |
| | | ACGATGGCCTGTACCAGGGGCTGAGCACAGCAACCAAAGATACTTATGAC |
| | | GCACTGCACATGCAGGCACTGCCACCCAGATGACAGCCAGGGGATTTCAC |
| | | CACTCAAAGGCCAGACCTGCAGACGCCCAGATTATGAGACACACTCGAG |
| CD8 signal | SEQ ID NO: 11 | MALPVTALLLPLALLLHAARP |
| peptide | | |
| VL-VH linker | SEQ ID NO: 12 | GGGGSGGGGSGGGGS |
| ER-retention | SEQ ID NO: 13 | GGGGSGGGGSGGGGSGGGGSAEKDEL |
| domain | | |
| ER-retention | SEQ ID NO: 14 | LYKYKSRRSFIEEKKMP |
| domain | | |
| CD8a hinge | SEQ ID NO: 15 | TTTPAPRPPTPAPTIASQPLSLRPEACRPAAGGAVHTRGLDFACDIYIWA |
| and | | PLAGTCGVLLLSLITLY |
| transmembrane | | |
| domain | | |
| 4-1 BB | SEQ ID NO: 16 | KRGRKKLLYIFKQPFMRPVQTTQEEDGCSCRFPEEEEGGCEL |
| intracellular | | |
| signaling | | |
| domain | | |
| CD3ζ | SEQ ID NO: 17 | RVKFSRSADAPAYQQGQNQLYNELNLGRREEYDVLDKRRGRDPEMGGKPR |
| intracellular | | RKNPQEGLYNELQKDKMAEAYSEIGMKGERRRGKGHDGLYQGLSTATKDT |
| signaling | | YDALHMQALPPR |
| domain | | |
| Anti-CD2 VH | SEQ ID NO: 18 | QVQLQQPGTELVRPGSSVKLSCKASGYTFTSYWVNWVKQRPDQGLEWIGR |
| (9-1) | | IDPYDSETHYNQKFTDKAISTIDTSSNTAYMQLSTLTSDASAVYYCSRSP |
| | | RDSSTNLADWGQGTLVTVSS |
| Anti-CD2 VL | SEQ ID NO: 19 | IVMTQSPATLSVTPGDRVSLSCRASQSISDYLHWYQQKSHESPRLLIKYA |
| (9-1) | | SQSISGIPSRFSGSGSGSDFTLSINSVEPEDVGVYYCQNGHSFPLTFGAG |
| | | TKLELRR |
| Anti-CD2 VH | SEQ ID NO: 20 | QLQQPGAELVRPGSSVKLSCKASGYTFTRYWIHWVKQRPIQGLEWIGNID |
| (9.6) | | PSDSETHYNQKFKDKATLTVDKSSGTAYMQLSSLTSEDSAVYYCATEDLY |
| | | YAMEYWGQGTSVTVSS |
| Anti-CD2 VL | SEQ ID NO: 21 | NIMMTQSPSSLAVSAGEKVTMTCKSSQSVLYSSNQKNYLAWYQQKPGQSP |
| (9.6) | | KLLIYWASTRESGVPDRFTGSGSGTDFTLTISSVQPEDLAVYYCHQYLSS |
| | | HTFGGGTKLEIKR |
| Anti-CD2 scFv | SEQ ID NO: 22 | IVMTQSPATLSVTPGDRVSLSCRASQSISDYLHWYQQKSHESPRLLIKYA |
| (9-1) | | SQSISGIPSRFSGSGSGSDFTLSINSVEPEDVGVYYCQNGHSFPLTFGAG |
| | | TKLELRRGGGGSGGGGSGGGGSQVQLQQPGTELVRPGSSVKLSCKASGYT |
| | | FTSYWVNWVKQRPDQGLEWIGRIDPYDSETHYNQKFTDKAISTIDTSSNT |
| | | AYMQLSTLTSDASAVYYCSRSPRDSSTNLADWGQGTLVTVSS |
| Anti-CD2 scFv | SEQ ID NO: 23 | NIMMTQSPSSLAVSAGEKVTMTCKSSQSVLYSSNQKNYLAWYQQKPGQSP |
| (9.6) | | KLLIYWASTRESGVPDRFTGSGSGTDFTLTISSVQPEDLAVYYCHQYLSS |
| | | HTFGGGTKLEIKRGGGGSGGGGSGGGGSQLQQPGAELVRPGSSVKLSCKA |
| | | SGYTFTRYWIHWVKQRPIQGLEWIGNIDPSDSETHYNQKFKDKATLTVDK |
| | | SSGTAYMQLSSLTSEDSAVYYCATEDLYYAMEYWGQGTSVTVSS |

It has been shown that secretion of cytokines by activated immune cells triggers cytokine release syndrome and macrophage activation syndrome, presenting serious adverse effects of immune cell therapy (Lee et al., Blood, 2014, 124(2): 188-195).

In some embodiments, the target-binding molecule is a molecule that specifically binds to a CD protein, e.g., human CD protein. In some cases, the target-binding molecule is an anti-CD2 antibody or an antigen-binding fragment that binds CD2.

All such suitable binding molecules capable of activating or inactivating an immune response upon binding to a ligand (e.g., peptide or antigen) expressed on a T cell are collectively referred to as a “target-binding molecule.” As would be appreciated by those of skill in the art, a target-binding molecule need not contain an antibody or antigen-binding fragment (e.g., scFv); rather the portion of the target-binding molecule that binds to a target molecule can be derived from, e.g., a receptor in a receptor-ligand pair, or a ligand in a receptor-ligand pair.

The target binding molecule of the PEBL described herein can be derived from an antibody that binds the target cell surface molecule. In some cases, the target binding molecule of the PEBL can be derived from an antibody that binds CD2. In some embodiments, the antibody that binds CD2 is the anti-CD2 monoclonal antibody 9.6 or a variant thereof. In some embodiments, the antibody that binds CD2 is a humanized variant of the anti-CD2 monoclonal antibody 9.6. In other embodiments, the antibody that binds CD2 is the anti-CD2 monoclonal antibody 9-1. In some embodiments, the antibody that binds CD2 is a humanized variant of the anti-CD2 monoclonal antibody 9-1.

Detailed descriptions of 9.6 and 9-1 can be found, e.g., in Kamoun et al. J Exp Med, 1981, 153:207-212 and in Bernard et al., in Leukocyte Typing II, 1986, eds. Reinherz, E. L., Haynes, B. F., Nadler, L. M., & Bernstein, I.D. (Springer, New York), pp. 53-66, respectively.

A humanized antibody refers to an immunoglobulin amino acid sequence variant or fragment thereof which is capable of binding to a target antigen (e.g., human CD2) and which comprises a framework (FR) region having substantially the amino acid sequence of a human immunoglobulin and a CDR having substantially the amino acid sequence of a non-human immunoglobulin. As such, a humanized antibody 9-1 can bind to CD2 and which comprises a FR region having substantially the amino acid sequence of a human immunoglobulin and a CDR having substantially the amino acid sequence of a murine antibody 9-1. Likewise, a humanized antibody 9.6 can bind to CD2 and which comprises a FR region having substantially the amino acid sequence of a human immunoglobulin and a CDR having substantially the amino acid sequence of a murine antibody 9.6.

In general, the humanized antibody comprise substantially all of at least one, and typically two, variable domains (Fab, Fab′, F(ab′)₂, Fabc, Fv) in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain.

The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody can comprise sequences from more than one class or isotype, and selecting particular constant domains to optimize desired effector functions is within the ordinary skill in the art.

The FR and CDR regions of the humanized antibody need not correspond precisely to the parental sequences, e.g., the import CDR or the consensus FR may be mutagenized by substitution, insertion or deletion of at least one residue so that the CDR or FR residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%.

In general, humanized antibodies are produced by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen.

Residues that influence antigen binding (e.g., cell surface molecule binding) are defined to be residues that are substantially responsible for the antigen affinity or antigen specificity of a candidate immunoglobulin, in a positive or a negative sense. In some cases, selection and combination of FR residues from the consensus and import sequence is performed to obtain the desired immunoglobulin characteristics. Such desired characteristics include increases in affinity and greater specificity for the target antigen, although it is conceivable that in some circumstances the opposite effects might be desired. In general, the CDR residues are directly and most substantially involved in influencing antigen binding (although not all CDR residues are so involved and therefore need not be substituted into the consensus sequence). However, FR residues also have a significant effect and can exert their influence in at least three ways: They may noncovalently directly bind to antigen, they may interact with CDR residues and they may affect the interface between the heavy and light chains.

Typically, it is necessary to impute the position of antigen from the spatial location of neighboring CDRs and the dimensions and structure of the target antigen. In general, only those humanized antibody residues that are capable of forming salt bridges, hydrogen bonds, or hydrophobic interactions are likely to be involved in non-covalent antigen binding, however residues which have atoms which are separated from antigen spatially by 3.2 Angstroms or less may also non-covalently interact with antigen. Such residues typically are the relatively larger amino acids having the side chains with the greatest bulk, such as tyrosine, arginine, and lysine. Antigen-binding FR residues also typically will have side chains that are oriented into an envelope surrounding the solvent oriented face of a CDR which extends about 7 Angstroms into the solvent from the CDR domain and about 7 Angstroms on either side of the CDR domain, again as visualized by three dimensional modeling.

A residue that interacts with a CDR generally is a residue that either affects the conformation of the CDR polypeptide backbone or forms a noncovalent bond with a CDR residue side chain. Conformation-affecting residues ordinarily are those that change the spatial position of any CDR backbone atom by more than about 0.2 Angstroms. Backbone atoms of CDR sequences are displaced for example by residues that interrupt or modify organized structures such as beta sheets, helices or loops. Residues that can exert a profound effect on the conformation of neighboring sequences include proline and glycine, both of which are capable of introducing bends into the backbone. Other residues that can displace backbone atoms are those that are capable of participating in salt bridges and hydrogen bonds.

A residue that interacts with a CDR side chain is one that is reasonably expected to form a noncovalent bond with a CDR side chain, generally either a salt bridge or hydrogen bond. Such residues are identified by three dimensional positioning of their side chains. A salt or ion bridge could be expected to form between two side chains positioned within about 2.5-3.2 Angstroms of one another that bear opposite charges, for example a lysinyl and a glutamyl pairing. A hydrogen bond could be expected to form between the side chains of residue pairs such as seryl or threonyl with aspartyl or glutamyl (or other hydrogen accepting residues). Such pairings are well known in the protein chemistry art and will be apparent to one skilled in the art upon three dimensional modeling of the candidate antibody.

Since it is not entirely possible to predict in advance what the exact impact of a given substitution will be it may be necessary to make the substitution and assay the candidate antibody for the desired characteristic. These steps, however, are per se routine and well within the ordinary skill of the art.

CDR and FR residues are determined according to a standard sequence definition (Kabat et al., Sequences of Proteins of Immunological Interest, National Institutes of Health, Bethesda Md. (1987), and a structural definition (as in Chothia and Lesk, J. Mol Biol. 196:901-917 (1987). Where these two methods result in slightly different identifications of a CDR, the structural definition is preferred, but the residues identified by the sequence definition method are considered important FR residues for determination of which framework residues to import into a consensus sequence.

Generally, the first step in humanizing an import antibody is deriving a consensus amino acid sequence into which to incorporate the import sequences. Next a model is generated for these sequences using the methods described above. In certain embodiments, the consensus human sequences are derived from the most abundant subclasses in the sequence compilation of Kabat et al. (Kabat, E. A. et al., Sequences of Proteins of Immunological Interest (National Institutes of Health, Bethesda, Md., 1987)). While these steps may be taken in different order, typically a structure for the candidate humanized antibody is created by transferring the at least one CDR from the non-human, import sequence into the consensus human structure, after the entire corresponding human CDR has been removed. The humanized antibody may contain human replacements of the non-human import residues at positions within CDRs as defined by sequence variability (Kabat, E. A. et al., Sequences of Proteins of Immunological Interest (National Institutes of Health, Bethesda, Md., 1987)) or as defined by structural variability (Chothia, C. & Lesk, A. M., J. Mol. Biol. 196:901-917 (1987)). Differences between the non-human import and the human consensus framework residues are individually investigated to determine their possible influence on CDR conformation and/or binding to antigen. Investigation of such possible influences is desirably performed through modeling, by examination of the characteristics of the amino acids at particular locations, or determined experimentally through evaluating the effects of substitution or mutagenesis of particular amino acids.

In some embodiments, a humanized antibody is made comprising amino acid sequence of an import, non-human antibody and a human antibody, utilizing the steps of: (a) obtaining the amino acid sequences of at least a portion of an import antibody variable domain and of a consensus human variable domain; (b) identifying Complementarity Determining Region (CDR) amino acid sequences in the import and the human variable domain sequences; (c) substituting an import CDR amino acid sequence for the corresponding human CDR amino acid sequence; (d) aligning the amino acid sequences of a Framework Region (FR) of the import antibody and the corresponding FR of the consensus antibody; (e) identifying import antibody FR residues in the aligned FR sequences that are non-homologous to the corresponding consensus antibody residues; (f) determining if the non-homologous import amino acid residue is reasonably expected to have at least one of the following effects: (1.) non-covalently binds antigen directly, (2.) interacts with a CDR; or (3.) participates in the VL-VH interface; and (g) for any non-homologous import antibody amino acid residue which is reasonably expected to have at least one of these effects, substituting that residue for the corresponding amino acid residue in the consensus antibody FR sequence.

Optionally, one determines if any non-homologous residues identified in step (e) are exposed on the surface of the domain or buried within it, and if the residue is exposed but has none of the effects identified in step (f), one may retain the consensus residue.

Additional descriptions of methods for generating humanized antibodies are found, for example, in U.S. Pat. Nos. 6,054,297; 6,407,213; and 6,719,971, the contents are incorporated herein by reference in their entireties.

In some embodiments, the target binding molecule of the PEBL comprises an anti-CD2 single chain variable fragment comprising a VH domain having at least 90% (e.g., 90%, 91%, 92%, 93%, 94%, 95%, 96%, 97%, 98%, 99%, or 100%) sequence identity to SEQ ID NO:18 and a VL domain having at least 90% (e.g., 90%, 91%, 92%, 93%, 94%, 95%, 96%, 97%, 98%, 99%, or 100%) sequence identity to SEQ ID NO:19. In some instances, a linker connects the VH domain and the VL domain of the scFv. The VH-VL linker can be a (GGGGS)ₙ (SEQ ID NO:35) linker where n can range from 1 to 6, e.g., 1, 2, 3, 4, 5, or 6. In other instances, the VH-VL linker can be any GS linker or other flexible linker known to one skilled in the art. In some instances, the VH domain comprises at least one (e.g., 1, 2, 3, 4, 5, 6, 7, 8, 9, 10, or more) amino acid substitution in the sequence set forth in SEQ ID NO:18. In some cases, the VL domain comprises at least one (e.g., 1, 2, 3, 4, 5, 6, 7, 8, 9, 10, or more) amino acid substitution in the sequence set forth in SEQ ID NO:19.

In some embodiments, the target binding molecule of the PEBL comprises an anti-CD2 single chain variable fragment comprising a VH domain having at least 90% (e.g., 90%, 91%, 92%, 93%, 94%, 95%, 96%, 97%, 98%, 99%, or 100%) sequence identity to SEQ ID NO:20 and a VL domain having at least 90% (e.g., 90%, 91%, 92%, 93%, 94%, 95%, 96%, 97%, 98%, 99%, or 100%) sequence identity to SEQ ID NO:21. In some instances, a linker connects the VH domain and the VL domain of the scFv. The VH-VL linker can be a (GGGGS)ₙ (SEQ ID NO:35) linker, where n can range from 1 to 6, e.g., 1, 2, 3, 4, 5, or 6. In other instances, the VH-VL linker can be any GS linker or other flexible linker known to one skilled in the art.

In some cases, anti-CD2 scFv comprises one or more amino acid substitutions that are compatible for binding to CD2 in human immune cells. In some embodiments, the VH domain comprises at least one (e.g., 1, 2, 3, 4, 5, 6, 7, 8, 9, 10, or more) amino acid substitution in the sequence set forth in SEQ ID NO:18 and the VL domain comprises at least one (e.g., 1, 2, 3, 4, 5, 6, 7, 8, 9, 10, or more) amino acid substitution in the sequence set forth in SEQ ID NO:19 such that the CD2 expression is blocked, reduced, or decreased in a human immune cell. In other embodiments, the VH domain comprises at least one (e.g., 1, 2, 3, 4, 5, 6, 7, 8, 9, 10, or more) amino acid substitution in the sequence set forth in SEQ ID NO:20 and the VL domain comprises at least one (e.g., 1, 2, 3, 4, 5, 6, 7, 8, 9, 10, or more) amino acid substitution in the sequence set forth in SEQ ID NO:21 such that the CD2 expression is blocked, reduced or decreased in a human immune cell.

In various embodiments, the target binding molecule of the PEBL described herein comprises an anti-CD2 scFv comprising at least 90% (e.g., 90%, 91%, 92%, 93%, 94%, 95%, 96%, 97%, 98%, 99%, or 100%) sequence identity to SEQ ID NO:22. In various other embodiments, the target binding molecule comprises an anti-CD2 scFv comprising at least 90% (e.g., 90%, 91%, 92%, 93%, 94%, 95%, 96%, 97%, 98%, 99%, or 100%) sequence identity to SEQ ID NO:23. In some embodiments, the anti-CD2 scFv is a variant of SEQ ID NO:22 and has the same binding activity as the anti-CD2 scFv of SEQ ID NO:22. In other embodiments, the anti-CD2 scFv is a variant of SEQ ID NO:23 and has the same binding activity as the anti-CD2 scFv of SEQ ID NO:23.

In some embodiments, the scFv of the PEBL comprises a variable heavy chain sequence having at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to a variable heavy chain sequence of an anti-CD2 antibody. In some embodiments, the scFv of the present invention comprises a variable light chain sequence having at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to a variable light chain sequence of an anti-CD2 antibody. For instance, the anti-CD2 antibody can be any such recognized by one skilled in the art. In some embodiments, the anti-CD2 antibody or anti-CD2 scFv of the invention is a humanized variant thereof.

In some embodiments, the anti-CD2 scFv of the PEBL binds intracellular CD2 in resting T cells and activated T cells. In other embodiments, the anti-CD2 scFv binds intracellular CD2 in activated T cells. In certain embodiments, the anti-CD2 scFv binds intracellular CD2 in activated T cells, and not resting T cells. In certain embodiments, the anti-CD2 scFv binds intracellular CD2 in an immune cell.

In some embodiments, the anti-CD2 scFv inhibits or blocks CD2 binding of CD58. In other embodiments, the anti-CD2 scFv does not block or inhibit CD2 binding of CD58.

The cellular localizing domain comprises a retention signaling domain. In some embodiments, the cellular localizing domain comprises a retention signaling domain and a transmembrane domain. In some instances, the cellular localizing domain comprises an endoplasmic reticulum (ER) retention sequence, a Golgi retention sequence, or a proteasome localizing sequence. The cellular localizing domain can include an amino acid sequence that prevents or hinders a protein from being secreted by a cell. The cellular localizing domain can include an amino acid sequence that retains a protein in an intracellular compartment. In some cases, the cellular localizing domain can include an amino acid sequence that retains an anchor a protein in a cellular membrane such as a membrane of the ER or Golgi. For instance, the retention signaling domain can contain a KDEL sequence (SEQ ID NO:24), KKD/E sequence (SEQ ID NO:25), KKXX sequence (SEQ ID NO:26), KKMP sequence (SEQ ID NO:27), or YQRL sequence (SEQ ID NO:28), wherein X represents any amino acid sequence. In some embodiments, if the retention signaling domain comprises KKXX or KKMP, the cellular localizing domain further comprises a CD8α hinge and transmembrane domain, such as but not limited to the sequence of SEQ ID NO:15. In some cases, the CD8α hinge and transmembrane domain is linked to the N-terminus of the ER retention domain of SEQ ID NO:14.

In some embodiments, the cellular localizing domain comprises a KDEL sequence (SEQ ID NO:24). In certain embodiments, the cellular localizing domain comprises the sequence of AEKDEL (SEQ ID NO:29). In various embodiments, the cellular localizing domain comprises the sequence of GGGGSGGGGSGGGGSGGGGSAEKDEL (SEQ ID NO:30). In particular embodiments, the cellular localizing domain comprises a KKMP sequence (SEQ ID NO:27). In some embodiments, the cellular localizing domain comprises the sequence of LYKYKSRRSFIEEKKMP (SEQ ID NO:31) and a KKMP sequence (SEQ ID NO:27).

In certain embodiments, the cellular localizing domain includes at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:30, as long as it possesses the desired function. In some embodiments, the cellular localizing domain includes at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:31, as long as it possesses the desired function.

In some embodiments, the ER retention sequence comprises an amino acid sequence selected from the group consisting of KDEL, KKXX, KKMP, and KKTN, wherein X can be any amino acid. In some embodiments, the Golgi retention sequence is selected from the group consisting of YGRL (SEQ ID NO:40), YQRL (SEQ ID NO:41), YKGL (SEQ ID NO:42), and YXXL (SEQ ID NO:43), wherein X can be any amino acid.

In some embodiments, proteasome localization is achieved by linking the scFv sequence to a tripartite motif containing 21 (TRIM21) targeting domain sequence and coexpressing the sequence encoding the human TRIM21 E3 ubiquitin ligase protein. TRIM21 binds with high affinity to the Fc domains of antibodies and can recruit the ubiquitin-proteosome complex to degrade molecules (e.g., proteins and peptides) bound to the antibodies. The TRIM21 targeting domain sequence encodes amino acid sequences selected from the group of human immunoglobulin G (IgG) constant regions (Fc) genes such as IgG1, IgG2, or IgG4 and is used to form a fusion protein comprising scFv and Fc domains. In this embodiment, the exogenously expressed TRIM21 protein binds the scFv-Fc fusion protein bound to the target protein (e.g., CD2) and directs the complex to the proteasome for degradation.

Details of the amino acid sequence of the human TRIM21 E3 ligase protein can be found, for example, in NCBI Protein database under NCBI Ref. Seq. No. NP 003132.2. Details of the nucleic acid sequence encoding the human TRIM21 E3 ligase protein can be found, for example, in NCBI Protein database under NCBI Ref. Seq. No. NM_003141.3.

The transmembrane domain can comprise a transmembrane domain or a combination of a hinge and transmembrane domain derived from CD8α, CD8β, 4-IBB, CD28, CD34, CD4, FcεRIγ, CD16, OX40, CD3ζ, CD3ε, CD3γ, CD3δ, TCRα, CD32, CD64, ICOS, VEGFR2, FAS, or FGFR2B. In certain embodiments, the transmembrane domain is derived from CD8α. In certain embodiments, the transmembrane domain is derived from CD8α. The hinge and transmembrane domain derived from CD8α are linked to an ER or Golgi retention signaling domain.

In some embodiments, the transmembrane domain or the hinge and transmembrane domain includes at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:15, as long as it possesses the desired function.

In some embodiments, the transmembrane domain is linked to the retention signaling domain by way of a linker. In some embodiments, the VL domain and VH domain of the scFv are connected by way of a linker. The linker between transmembrane domain and the retention signaling domain is the same sequence of the linker of the scFv. In some instances, the linker between transmembrane domain and the retention signaling domain has a different sequence than the linker of the scFv. Non-limiting examples of a linker include (GS)ₙ, (GGS)ₙ, (GGGS)ₙ (SEQ ID NO:32), (GGSG)ₙ (SEQ ID NO:33), (GGSGG)ₙ (SEQ ID NO:34), or (GGGGS)ₙ (SEQ ID NO:35), wherein n is 1, 2, 3, 4, 5, 6, 7, 8, 9, or 10. In some embodiment, the linker is (GGGGS)₃ (SEQ ID NO:36) or (GGGGS)₄ (SEQ ID NO:37). Variation in the linker length may retain or enhance activity, giving rise to superior efficacy in activity studies.

In particular embodiments, the linker comprises, e.g., GGGGSGGGGS (SEQ ID NO:38). In some embodiments, the linker comprises, e.g., GGGGSGGGGSGGGGSGGGGS (SEQ ID NO:39). In various embodiments, peptide linkers having lengths of about 5 to about 100 amino acids, inclusive, can be used in the present invention. In certain embodiments, peptide linkers having lengths of about 20 to about 40 amino acids, inclusive, can be used in the present invention. In some embodiments, peptide linkers having lengths of at least 5 amino acids, at least 10 amino acids, at least 15 amino acids, at least 20 amino acids, at least 25 amino acids, at least 30 amino acids, at least 35 amino acids, or at least 40 amino acids can be used in the present invention. As would be appreciated by those of skill in the art, such linker sequences as well as variants of such linker sequences are known in the art. Methods of designing constructs that incorporate linker sequences as well as methods of assessing functionality are readily available to those of skill in the art.

In particular embodiments, the signal peptide is derived from a CD8α signaling peptide. In some embodiments, the signal peptide comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to SEQ ID NO:11. The signal peptide can be located N-terminal to the target-binding molecule.

As those skilled in the art would appreciate, in certain embodiments, any of the sequences of the various components disclosed herein (e.g., signal peptide, scFv, intracellular signaling domain, transmembrane domain, linker, localizing domain, and combinations thereof) can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the specific corresponding sequences disclosed herein.

Exemplary embodiments of PEBLs described herein are provided in Table 1.

In some embodiments, the nucleic acid sequence encoding an anti-CD2 PEBL comprises one or more nucleic acid sequences set forth in Table 1. In certain embodiments, the anti-CD2 PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:6, or a codon optimized variant thereof. In certain embodiments, the anti-CD2 PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:7, or a codon optimized variant thereof. In some embodiments, the anti-CD2 PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:8, or a codon optimized variant thereof. In other embodiments, the anti-CD2 PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:9, or a codon optimized variant thereof. For instance, the nucleic acid sequence encoding the anti-CD2 PEBL can be modified to obtain to desired expression or activity in human cells, e.g., human immune cells.

Methods of producing antibodies and antibody fragments thereof against any target protein are well-known and routine in the art. Moreover, as exemplified herein, commercially available antibodies to various targets, e.g., CD2 can be used to generate a PEBL molecule, as exemplified herein. Antibodies known in the art, as well as fragments of antibodies (e.g., scFv) derived therefrom, can be used in the present invention, as exemplified herein.

As would be appreciated by those of skill in the art, the chimeric antigen receptor and/or the PEBL molecule can be designed to bind to the targets disclosed herein, as well as variants of the targets disclosed herein. By way of example, a chimeric antigen receptor and/or the PEBL molecule can be designed to bind to a CD2, or a naturally-occurring variant molecule thereof. Such naturally-occurring variants can have the same function as the wild-type form of the molecule. In other embodiments, the variant can have a function that is altered relative to the wild-type form of the molecule (e.g., confers a diseased state).

As would be appreciated by those of skill in the art, the various components of the PEBL molecule constructs can be substituted in different combinations (e.g., to contain a different linker, different localizing sequence, different scFv, etc.), so long as the combination produces a functional PEBL. Methods of assessing functionality for a particular construct are within the ambit of those of skill in the art, as disclosed herein.

### IV. Chimeric Antigen Receptors

Chimeric Antigen Receptors (CARs) are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. The signaling domains for first generation CARs have been derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX40 (CD134), and 4-1BB (CD137) have been added alone (second generation) or in combination (third generation) to enhance survival and increase proliferation of CAR modified T cells.

In addition to single-chain CARs, in some embodiments, the CARs described herein are multi-chain CARs. Multi-chain CARs or multi-specific CARs comprise several (e.g., two or more) extracellular antigen-(ligand)-binding domains, to simultaneously bind different targets, thereby augmenting immune cell activation and function. In some instances, the extracellular antigen-binding domains are placed in tandem on the same transmembrane polypeptide, and optionally can be separated by a linker. In other instances, the different extracellular antigen-binding domains can be placed on different transmembrane polypeptides composing the multi-chain CAR. Similar to a single-chain CAR, the signal transducing domain of a multi-chain CAR can be the cytoplasmic sequences of the Fc receptor or T cell receptor and co-receptors that act in concert to initiate signal transduction following antigen receptor engagement, as well as any derivate or variant of these sequences and any synthetic sequence that as the same functional capability.

A signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs (ITAMs). Non-limiting examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. A signal transduction domain can also include a co-stimulatory signal molecule. Additional description of bispecific or multispecific CARs are described in WO2014/4011988, the contents are incorporated by reference in their entirety.

Accordingly, in one embodiment, the present invention relates to an engineered immune cell that comprises a nucleic acid comprising a nucleotide sequence encoding a chimeric antigen receptor (e.g., CAR), and a nucleic acid comprising a nucleotide sequence encoding a target-binding molecule linked to a localizing domain (e.g., PEBL) that binds CD2. In some embodiments, the present invention relates to an engineered immune cell (such as a CAR-T cell) comprises a chimeric antigen receptor (e.g., CAR) that binds CD2 and a target-binding molecule linked to a localizing domain (e.g., PEBL) that binds CD2. The CD2 CAR of the CAR-T cell binds CD2 on the cell surface of another cell, and the CD2 PEBL the CAR-T cell binds CD2 located in the intracellular compartment of the CAR-T cell. As such, the CD2 PEBL prevents fratricide of CAR-T cells by other CD2 binding CARs.

In certain aspects of the present invention, the chimeric antigen receptor (CAR) binds to a CD molecule, e.g., CD2 that is expressed on the surface of a cell. In other embodiments, the CAR binds to CD3, CD4, CD5, CD7, CD8, CD25, CD28, CD30, CD38, CD45, CD45RA, CD45RO, CD52, CD56, CD57, CD99, CD127, or CD137.

The CD2 binding domain of the CAR can be an anti-CD2 antibody or an antigen-binding fragment that binds CD2. In some embodiments, the antibody that binds CD2 is the anti-CD2 monoclonal antibody 9.6. In other embodiments, the antibody that binds CD2 is the anti-CD2 monoclonal antibody 9-1. In some embodiments, the antibody that binds CD2 is the anti-CD2 monoclonal antibody 9.6 or a variant thereof. In some embodiments, the antibody that binds CD2 is a humanized variant of the anti-CD2 monoclonal antibody 9.6. In other embodiments, the antibody that binds CD2 is the anti-CD2 monoclonal antibody 9-1. In some embodiments, the antibody that binds CD2 is a humanized variant of the anti-CD2 monoclonal antibody 9-1.

In some embodiments, the CD2 binding domain is an anti-CD2 scFv. In some embodiments, the scFv of the present invention comprises a variable heavy chain sequence having at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to a variable heavy chain sequence of an anti-CD2 antibody. In some embodiments, the scFv of the present invention comprises a variable light chain sequence having at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to a variable light chain sequence of an anti-CD2 antibody. For instance, the anti-CD2 antibody can be any such recognized by one skilled in the art.

In some embodiments, the anti-CD2 single chain variable fragment can contain a VH domain having at least 90% (e.g., 90%, 91%, 92%, 93%, 94%, 95%, 96%, 97%, 98%, 99%, or 100%) sequence identity to SEQ ID NO: 18 and a VL domain having at least 90% (e.g., 90%, 91%, 92%, 93%, 94%, 95%, 96%, 97%, 98%, 99%, or 100%) sequence identity to SEQ ID NO: 19. In some instances, a linker connects the VH domain and the VL domain of the scFv. The VH-VL linker can be a (GGGGS)ₙ (SEQ ID NO:35) linker where n can range from 1 to 6, e.g., 1, 2, 3, 4, 5, or 6. The VH-VL linker can be a (GGGGS)ₙ (SEQ ID NO:35) linker where n can range from 1 to 6, e.g., 1, 2, 3, 4, 5, or 6. In other instances, the VH-VL linker can be any GS linker or other flexible linker known to one skilled in the art.

In some instances, the VH domain comprises at least one (e.g., 1, 2, 3, 4, 5, 6, 7, 8, 9, 10, or more) amino acid substitution in the sequence set forth in SEQ ID NO:18. In some cases, the VL domain comprises at least one (e.g., 1, 2, 3, 4, 5, 6, 7, 8, 9, 10, or more) amino acid substitution in the sequence set forth in SEQ ID NO:19.

In some embodiments, the anti-CD2 scFv contains a VH domain having at least 90% (e.g., 90%, 91%, 92%, 93%, 94%, 95%, 96%, 97%, 98%, 99%, or 100%) sequence identity to SEQ ID NO: 20 and a VL domain having at least 90% (e.g., 90%, 91%, 92%, 93%, 94%, 95%, 96%, 97%, 98%, 99%, or 100%) sequence identity to SEQ ID NO: 21. In some instances, a linker connects the VH domain and the VL domain of the scFv. The VH-VL linker can be a (GGGGS)ₙ (SEQ ID NO:35) linker where n can range from 1 to 6, e.g., 1, 2, 3, 4, 5, or 6. In other instances, the VH-VL linker can be any GS linker or other flexible linker known to one skilled in the art.

In some cases, anti-CD2 scFv comprises one or more amino acid substitutions that are compatible for binding to CD2 in human immune cells. In some embodiments, the VH domain comprises at least one (e.g., 1, 2, 3, 4, 5, 6, 7, 8, 9, 10, or more) amino acid substitution in the sequence set forth in SEQ ID NO:18 and the VL domain comprises at least one (e.g., 1, 2, 3, 4, 5, 6, 7, 8, 9, 10, or more) amino acid substitution in the sequence set forth in SEQ ID NO:19 such that the CD2 expression is blocked, reduced or decreased in a human immune cell. In other embodiments, the VH domain comprises at least one (e.g., 1, 2, 3, 4, 5, 6, 7, 8, 9, 10, or more) amino acid substitution in the sequence set forth in SEQ ID NO:20 and the VL domain comprises at least one (e.g., 1, 2, 3, 4, 5, 6, 7, 8, 9, 10, or more) amino acid substitution in the sequence set forth in SEQ ID NO:21 such that the CD2 expression is blocked, reduced or decreased in a human immune cell.

In various embodiments, the anti-CD2 scFv comprises at least 90% (e.g., 90%, 91%, 92%, 93%, 94%, 95%, 96%, 97%, 98%, 99%, or 100%) sequence identity to SEQ ID NO:22. In various other embodiments, the anti-CD2 scFv comprises at least 90% (e.g., 90%, 91%, 92%, 93%, 94%, 95%, 96%, 97%, 98%, 99%, or 100%) sequence identity to SEQ ID NO:23. In some embodiments, the anti-CD2 scFv is a variant of SEQ ID NO:22 and has the same binding activity as the anti-CD2 scFv of SEQ ID NO:22. In other embodiments, the anti-CD2 scFv is a variant of SEQ ID NO:23 and has the same binding activity as the anti-CD2 scFv of SEQ ID NO:23.

The CD2 binding domain of the PEBL can bind the same epitope of CD2 as the anti-CD2 binding domain of the CAR. In other cases, the anti-CD2 binding domain of the PEBL can bind a different epitope of CD2 than the CD2 binding domain of the CAR. The amino acid sequences of the CD2 binding domain of the PEBL and the CD2 binding domain of the CAR can be substantially identical. Or, the amino acid sequences of the CD2 binding domain of the PEBL and the CD2 binding domain of the CAR can be different. In some embodiments, the sequence of the CD2 binding domain of the PEBL has at least 90% (e.g., 90%, 91%, 92%, 93%, 94%, 95%, 96%, 97%, 98%, 99%, or 100%) sequence identity to the CD2 binding domain of the CAR.

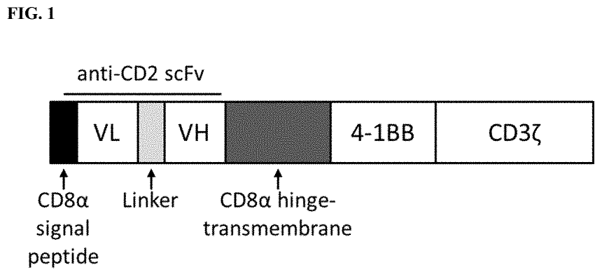

Exemplary embodiments of CARs are shown in FIG. 1 and exemplary amino acid and nucleic acid sequences are provided in Table 1.

In some embodiments, the nucleic acid sequence encoding an anti-CD2 CAR comprises one or more nucleic acid sequences set forth in Table 1. In certain embodiments, the anti-CD2 CAR comprises the nucleotide sequence having at least 90% sequence identity (e.g., 90%, 91%, 92%, 93%, 94%, 95%, 96%, 97%, 98%, 99%, or more sequence identity) to SEQ ID NO:10, or a codon optimized variant thereof. For instance, the nucleic acid sequence encoding the anti-CD2 CAR can be modified to obtain to desired expression or activity in human cells, e.g., human immune cells.

As those skilled in the art would appreciate, in certain embodiments, any of the sequences of the various components disclosed herein (e.g., signal peptide, scFv, intracellular signaling domain(s), transmembrane domain, linker, and combinations thereof) can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the specific corresponding sequences disclosed herein.

In some embodiments, the 4-1BB intracellular signaling domain can have at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:16, as long as it possesses the desired function.

In certain embodiments, the 4-1BB intracellular signaling domain can be replaced by another intracellular signaling domain from a co-stimulatory molecule such as CD28, OX40, ICOS, CD27, GITR, HVEM, TIM1, LFA1, or CD2. In some embodiments, the intracellular signaling domain of the CAR can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2. Optionally, the 4-1BB intracellular signaling domain can also include another intracellular signaling domain (or a portion thereof) from a co-stimulatory molecule such as CD28, OX40, ICOS, CD27, GITR, HVEM, TIM1, LFA1, or CD2. In some embodiments, the additional intracellular signaling domain can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2.

In some embodiments, the CD3zeta (CD3) intracellular signaling domain can have at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:17, as long as it possesses the desired function.

In some instances, the intracellular signaling domain comprises an immunoreceptor tyrosine-based activation motif (ITAM) or a portion thereof, as long as it possesses the desired function. The intracellular signaling domain of the CAR can include a sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to an ITAM. In certain embodiments, the intracellular signaling domain can have at least 95% sequence identity, at least 96% sequence identity, at least 97% sequence identity, at least 98% sequence identity, at least 99% sequence identity, or 100% sequence identity to FccRIy, CD4, CD7, CD8, CD28, OX40 or H2-Kb, as long as it possesses the desired function.

In some embodiments, the CD8alpha (CD8α) hinge and transmembrane domain can have at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:11, as long as it possesses the desired function.

Hinge and transmembrane sequences suitable for use in the present invention are known in the art, and provided in, e.g., publication WO2016/126213, incorporated by reference in its entirety.

In some embodiments, the hinge and transmembrane domain of the anti-CD2 CAR can include a signaling domain (e.g., transmembrane domain) from CD8α, IgG, CD8β, 4-IBB, CD28, CD34, CD4, FcεcRIγ, CD16, OX40, CD3, CD3ε, CD3γ, CD3δ, TCRα, CD32, CD64, VEGFR2, FAS, FGFR2B, or another transmembrane protein. The transmembrane domain may also be a non-naturally occurring hydrophobic protein segment.

In some embodiments, the CD8alpha (CD8α) signal peptide can have at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:11, as long as it possesses the desired function.

### V. Engineered Immune Cells

Provided herein is an engineered immune cell comprising a genetic modification of a target CD2 nucleic acid sequence, and a chimeric antigen receptor (CAR) that binds CD2. In some embodiments, the engineered cell also includes a blocking polypeptide. Such an engineered immune cell lacks endogenous CD2 expression and expresses a CAR that binds CD2.

In some embodiments, the genetic modification of the target CD2 nucleic acid sequence results in inactivation of CD2 expression. In some instances, the genetic modification comprises gene editing using a system selected from the group consisting of CRISPR/Cas, zinc finger nucleases, TALENs, and meganucleases. In some instances, the genetic modification may comprise gene silencing (such as transient gene silencing) using a system selected from the group consisting of short hairpin RNA, double-stranded RNA, sdRNA, short inhibitory RNA, and microRNA. In some embodiments, endogenous CD2 expression is blocked in the engineered immune cell. For instance, CD2 protein expression on the surface of a cell is blocked or prevented by genetic modification. In some cases, CD2 protein expression in the cell (e.g., in the intracellular environment of the cell) is blocked or prevented by genetic modification. In some embodiments, endogenous expression of CD2 protein is blocked or prevented in the engineered immune cell.

The blockage of endogenous CD2 expression may persist for at least 6 months. The blockage of endogenous CD2 expression may persist for at least 12 months. In some embodiments, the engineered immune cell proliferates at a substantially equivalent rate as a comparable immune cell.

Accordingly, in one embodiment, the present invention relates to an engineered immune cell that expresses a CD2 CAR and is genetically modified to lack endogenous CD2 surface expression. In certain embodiments, the engineered immune cell is an engineered T cell, an engineered natural killer (NK) cell, an engineered NK/T cell, an engineered monocyte, an engineered macrophage, or an engineered dendritic cell. In some embodiments, the immune cell is a peripheral blood mononuclear cell (PBMC)-derived T cell.

In some embodiments, the present invention describes an engineered immune cell expressing a PEBL that binds a cell surface or secreted molecule, such as those outlined herein. In some embodiments, the engineered immune cell also comprises a genetic modification of a target CD2 nucleic acid sequence. In some instances, the CD2 targeted genetic modification is a CRISPR-based genetic modification. In some embodiments, the CRISPR-based genetic modification of CD2 utilizes one or more guide RNA sequences of SEQ ID NOS:44-47. In some instances, the CD2 targeted genetic modification is a TALEN-based or ZFN-based genetic modification. In certain embodiments, the engineered immune cell is an engineered T cell, an engineered γδ T cell, a PBMC-derived T cell, an engineered natural killer (NK) cell, an engineered NK/T cell, an engineered monocyte, an engineered macrophage, or an engineered dendritic cell.

In some embodiments, the present invention is directed to an engineered immune cell expressing a CAR that binds CD2 includes those outlined herein. In some embodiments, the engineered cell expresses a CAR comprising the amino acid sequence having at least 90% (e.g., 90%, 91%, 92%, 93%, 94%, 95%, 96%, 97%, 98%, 99%, or more) identity of SEQ ID NO:5. In certain embodiments, the engineered cell expresses a CAR comprising the amino acid sequence of SEQ ID NO:5. In certain embodiments, the engineered immune cell is an engineered T cell, an engineered γδ T cell, a PBMC-derived T cell, an engineered natural killer (NK) cell, an engineered NK/T cell, an engineered monocyte, an engineered macrophage, or an engineered dendritic cell.

In some embodiments, the present invention is directed to an engineered immune cell expressing a CAR that binds CD2 and a PEBL that binds CD2, including those outlined herein. In some embodiments, the engineered immune cell expresses a PEBL comprising an amino acid sequence having at least 90% (e.g., 90%, 91%, 92%, 93%, 94%, 95%, 96%, 97%, 98%, 99%, or more) identity to any one of SEQ ID NOS:1-4 in addition to a CAR comprising the amino acid sequence having at least 90% (e.g., 90%, 91%, 92%, 93%, 94%, 95%, 96%, 97%, 98%, 99%, or more) identity to SEQ ID NO:5. In certain embodiments, the engineered immune cell expresses a PEBL comprising an amino acid sequence selected from the group consisting of any one of SEQ ID NOS:1-4 in addition to a CAR comprising the amino acid sequence of SEQ ID NO:5. In some embodiments, the engineered immune cell also comprises a genetic modification of a target CD2 nucleic acid sequence (e.g., an endogenous CD2 locus). In some instances, the CD2 targeted genetic modification is a CRISPR-based genetic modification. In some embodiments, the CRISPR-based genetic modification of CD2 utilizes one or more guide RNA sequences of SEQ ID NOS:44-47.

In some instances, the engineered immune cell expresses a PEBL having at least 90% identity to the amino acid sequence of SEQ ID NO:1 and a CAR having at least 90% identity to the amino acid sequence of SEQ ID NO:5. In some instances, the engineered immune cell expresses a PEBL having at least 90% identity to the amino acid sequence of SEQ ID NO:2 and a CAR having at least 90% identity to the amino acid sequence of SEQ ID NO:5. In some instances, the engineered immune cell expresses a PEBL having at least 90% identity to the amino acid sequence of SEQ ID NO:3 and a CAR having at least 90% identity to the amino acid sequence of SEQ ID NO:5. In some instances, the engineered immune cell expresses a PEBL having at least 90% identity to the amino acid sequence of SEQ ID NO:4 and a CAR having at least 90% identity to the amino acid sequence of SEQ ID NO:5. In some embodiments, the engineered immune cell expresses a PEBL of SEQ ID NO:1 and a CAR of SEQ ID NO:5. In some embodiments, the engineered immune cell expresses a PEBL of SEQ ID NO:1 and a CAR of SEQ ID NO:5. In some embodiments, the engineered immune cell expresses a PEBL of SEQ ID NO:2 and a CAR of SEQ ID NO:5. In some embodiments, the engineered immune cell expresses a PEBL of SEQ ID NO:3 and a CAR of SEQ ID NO:5. In some embodiments, the engineered immune cell expresses a PEBL of SEQ ID NO:4 and a CAR of SEQ ID NO:5. In some embodiments, such an engineered immune cell also contains a genetic modification of a target CD2 nucleic acid sequence. In some instances, the CD2 targeted genetic modification is a CRISPR-based genetic modification. In some embodiments, the CRISPR-based genetic modification of CD2 utilizes one or more guide RNA sequences of SEQ ID NOS:44-47. In certain embodiments, the engineered immune cell is an engineered T cell, an engineered γδ T cell, a PBMC-derived T cell, an engineered natural killer (NK) cell, an engineered NK/T cell, an engineered monocyte, an engineered macrophage, or an engineered dendritic cell.

PEBLs outlined herein prevent transport of target proteins to a cellular membrane. For instance, PEBLs directed to the target molecule described above are retained in the ER. Thus, expression of the target on the cell surface is suppressedIn some instances, the PEBLs do not cause immunophenotypic changes in the engineered immune cell. Also, the PEBLs do not affect proliferation of the engineered immune cell. In some embodiments, the PEBLs are co-expressed with a CAR, such as an anti-CD2-4-1BB-CD3ζ CAR.

In certain embodiments, provided is an engineered immune cell comprising: a nucleic acid comprising a nucleotide sequence encoding a target-binding molecule linked to a localizing domain (e.g., PEBL), wherein the target-binding molecule is an antibody that binds the target molecule, and the localizing domain comprises a retention signal domain comprising an amino acid sequence selected from the group consisting of an endoplasmic reticulum (ER) sequence, a Golgi retention sequence, and a proteosome localizing sequence. In some cases, the PEBL also includes a transmembrane domain sequence derived from CD8α, CD8β, 4-IBB, CD28, CD34, CD4, FcεRIγ, CD16, OX40, CD3ζ, CD3ε, CD3γ, CD3δ, TCRα, CD32, CD64, VEGFR2, FAS, or FGFR2B.

In some instances, the engineered cell comprises a nucleic acid comprising a nucleotide sequence encoding a chimeric antigen receptor (CAR). In certain cases, the CAR comprises intracellular signaling domains of 4-1BB and CD3, and an antibody that binds CD2. In certain embodiments, the antibody that binds CD2 in the context of the target-binding molecule comprises: a VH sequence and a VL sequence set forth in Table 1.

In some embodiments, the engineered immune cell comprises a genetic modification of a target CD2 nucleic acid sequence. In some instances, the CD2 targeted genetic modification is a CRISPR-based genetic modification. In some embodiments, the CRISPR-based genetic modification of CD2 utilizes one or more guide RNA sequences corresponding to the nucleic acid sequences of SEQ ID NOS:44-47.

In some embodiments, the engineered immune cell is an engineered T cell, an engineered natural killer (NK) cell, an engineered NK/T cell, an engineered monocyte, an engineered macrophage, or an engineered dendritic cell. In some cases, the engineered immune cell is an allogeneic cell. In other cases, the engineered immune cell is an autologous cell.

In some embodiments, the engineered immune cell lacks CD2 surface expression for at least 6 months. In other embodiments, the engineered immune cell lacks CD2 surface expression for at least 12 months. In particular embodiments, the engineered immune cell lacks CD2 surface expression for at least 20 months. In some embodiments, the engineered immune cell lacks CD2 surface expression for at least 24 months.

In certain embodiments, the engineered immune cell proliferates at a substantially equal rate compared to a comparable immune cell. In some embodiments, the engineered immune cell carrying a genetic modification of CD2 gene and expressing a CAR (e.g., CD2ΔCD2CAR-T cell) and proliferates similar to an immune cell expressing the corresponding CAR (e.g., CD2CAR-T cell).

In some embodiments, the engineered immune cells of the present invention have enhanced therapeutic efficacy. Such engineered immune cell can be used to treat a cancer in a subject. In certain embodiments, the cancer is a CD2 associated cancer or a T cell malignancy, e.g., T cell leukemia or T cell lymphoma, such a T-cell acute lymphoblastic leukemia (T-ALL), T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, cutaneous T-cell lymphomas (CTCL) and subtypes thereof, mycosis fungoides, Sézary syndrome, primary cutaneous gamma-delta T-cell lymphoma, malignancies with the T lineage subsets of Non-Hodgkin's lymphoma (NHL), including but not limited to, peripheral T-cell lymphoma (PTCL) not otherwise specified (PTCL-NOS), angioimmunoblastic T-cell lymphoma, and anaplastic large cell lymphoma. In certain embodiments, the T cell malignancy is early T-cell progenitor acute lymphoblastic leukemia (ETP-ALL).

In some embodiments, the engineered immune cells of the present invention carrying a genetic modification of CD2 gene, expressing a CAR, and expressing a PEBL (e.g., CD2Δ_PEBL_CD2CAR-T cell) has an enhanced or increased therapeutic effect compared to an immune cell expressing the corresponding CAR (e.g., CD2CAR-T cell). In some embodiments, the engineered immune cells expressing a CAR and an anti-CD2 PEBL have a comparable therapeutic effect as an immune cell expressing the corresponding CAR.

In another embodiment, the present invention relates to a method for producing an engineered immune cell of the present invention, comprising introducing into an immune cell a nucleic acid comprising a nucleotide sequence encoding a chimeric antigen receptor, and a nucleic acid comprising one or more nucleotide sequences for genetic modification of CD2. In some embodiments, the nucleotide sequences for genetic modification of CD2 comprises sequences encoding a Cas protein or a variant thereof. In some embodiments, the nucleotide sequence for genetic modification of CD2 comprises a guide RNA corresponding to any one selected from the group consisting of SEQ ID NOS:44-47. In some embodiments, the method also includes introducing a nucleic acid comprising a nucleotide sequence encoding a target-binding molecule linked to a localizing domain (e.g., a PEBL molecule), thereby producing an engineered immune cell.

In certain embodiments, the engineered immune cell is an engineered T cell, an engineered natural killer (NK) cell, an engineered NK/T cell, an engineered monocyte, an engineered macrophage, or an engineered dendritic cell. In some embodiments, the engineered T cell is any type of T cell. In certain embodiments, the engineered T cell is a gamma-delta (γδ) T cell. In certain embodiments, the engineered T cell is produced from a PBMC-derived T cell.

In certain embodiments, the nucleic acid comprising a nucleotide sequence is introduced into an immune cell ex vivo. In other embodiments, the nucleic acid comprising a nucleotide sequence is introduced into an immune cell in vivo.

The nucleic acid comprising a nucleotide sequence to be introduced can be a single bicistronic construct containing a chimeric antigen receptor described herein and a target-binding molecule (e.g., scFv) linked to a localizing domain. As described herein, a single bicistronic construct can be prepared by inserting an internal ribosomal entry site (IRES) or a 2A peptide-coding region site between the 2 cDNAs encoding the chimeric antigen receptor as described herein (e.g., CAR) and the target-binding molecule (e.g., scFv). The design of tricistronic delivery systems to delete more than one target should also be feasible. Alternatively, separate transductions (simultaneously or sequentially) of the individual constructs (e.g., CAR and PEBL) could be performed.

Methods of introducing exogenous nucleic acids are exemplified herein, and are well-known in the art. Any means known in the art to allow delivery inside cells or subcellular compartments of agents/chemicals and molecules (proteins and nucleic acids) can be used including liposomal delivery means, polymeric carriers, chemical carriers, lipoplexes, polyplexes, dendrimers, nanoparticles, emulsion, natural endocytosis or phagocytose pathway as non-limiting examples, as well as physical methods such as electroporation. In some embodiments, polynucleotides are transfected under mRNA form, which is introduced directly into the cells, for example by electroporation. In some embodiments, viral vectors comprising one or more of the polynucleotides described herein are an alternative to the standard means of transfection. Methods for viral transduction are well known in the art. In some embodiments, viral vectors, including but not limited to retroviral or lentiviral vectors are used for stable integration into the genome of the engineered cell.

In some embodiments, the nucleotide sequence encoding a CAR, the nucleotide sequence encoding a PEBL, and the nucleotide sequence(s) for the genetic modification of CD2 are introduced sequentially. In other embodiments, the nucleotide sequence encoding a CAR, the nucleotide sequence encoding a PEBL are introduced simultaneously, and the nucleotide sequence(s) for the genetic modification of CD2 are. In certain cases, the nucleotide sequence encoding a CAR and the nucleotide sequence encoding a PEBL are operatively linked, and thus can be introduced on a single expression vector or plasmid.

In some embodiments, the immune cells are cultured in the presence of one or more cytokines including, but not limited to, IL-2, IL-7, IL-15, and any combination thereof. In some cases, the immune cells are cultured in the presence of an agent capable of enhancing or inducing proliferation of T cells, CD4+ T cells and/or CD8+ T cell. In some cases, the immune cells are cultured in the presence of an agent that binds a molecule of the TCR/CD3 complex and/or an agent that binds CD28. In certain embodiments, the method of culturing the engineered immune cell includes culturing in the presence of a molecule selected from the group consisting of CD90 (Thy-1), CD95 (Apo−/Fas), CD137 (4-1BB), CD154 (CD40L), ICOS, LAT, CD27, OX40 and HVEM. In certain embodiments, the method of culturing includes culturing in the presence of an agent that binds to CD90 (Thy-1), CD95 (Apo−/Fas), CD137 (4-1BB), CD154 (CD40L), ICOS, LAT, CD27, OX40, or HVEM. Additional method for culturing the engineered immune cells described herein can be found in, e.g., US20190136186, US20190062706, and US20170037369.

In some embodiments, peripheral blood mononuclear cells (PBMCs) are obtained. In some embodiments, peripheral blood mononuclear cells (PBMCs) are harvested from a human subject. In some embodiments, peripheral blood mononuclear cells (PBMCs) are harvested from a healthy human subject. In some embodiments, peripheral blood mononuclear cells (PBMCs) are harvested from a human subject with a cancer, including any described herein. In some embodiments, positive selection of T cells is performed with either (a) CD3 microbeads, or (b) both CD4 and CD8 microbeads, in accordance with the manufacturer's recommendations. In some cases, cells are resuspended at 1×10⁷ cells per 80 μl of MACS buffer, comprising sterile filtered PBS+0.5% BSA+2 mM EDTA, and labelled with 20 μl of microbeads per 80 μl of cell suspension. Cells are incubated at 4° C. for 15 minutes, and then washed with MACS buffer. Labelled cells are passed through a LS column (Miltenyi Biotec), and positively selected T cells bound to the LS column are eluted into a collection tube. Isolated T cells are washed, and resuspended in TexMACS medium supplemented with 3% human AB serum (Sigma) at a density of 1×10⁶ cells per ml. In some embodiments, T cells are activated with 10 μl T Cell TransAct (Miltenyi Biotec) per 1×10⁶ T cells, and cultured with either (a) 120 IU/ml recombinant human IL-2, or (b) 12.5 ng/ml recombinant human IL-7 and 12.5 ng/ml recombinant human IL-15.

In some embodiments, one day after selection and activation (Day 1), T cells are transduced with lentivirus at MOI for 1-10 (e.g., MOI 1, MOI 2, MOI 3, MOI 4, MOI 5, MOI 6, MOI 7, MOI 8, MOI 9, and MOI 10) using static transduction. In some cases, the T cell cultures are monitored and maintained at a cell density of 0.5-2×10⁶ T cells per ml of culture media. Fresh IL-2, or IL-7 and IL-15 cytokines can be added to the cultures every 3-4 days. In some embodiments, ten days post transduction (Day 11), expanded T cells are harvested. In some cases the expanded T cells are analysed using functional assays and phenotypic analysis by flow cytometry.

In some embodiments, the lentivirus comprises one or more constructs for CD2 gene editing or gene silencing. In some embodiments, the lentivirus comprises one or more constructs for a CAR described herein. In some embodiments, the lentivirus comprises one or more constructs for a PEBL described herein. In some embodiments, CD2ΔCD2-CAR cells are generated with two or more sequential lentiviral transductions. In some embodiments, T cells are transduced with lentiviral vectors encoding the CD2 gene editing construct (e.g., CD2-CRISPR sgRNA construct) on a first day (Day 1), and followed by a second transduction with lentiviral vectors encoding the CD2-CAR construct three days after the first transduction (Day 4). The resulting transduced CD2ΔCD2-CAR cells can be harvested at about 5 to 10 days or more after the first transduction.

In various aspects, also provided is a kit for producing an engineered immune cell described herein. The present kit can be used to produce, e.g., allogeneic or autologous effector T cells.

Accordingly, provided herein is a kit comprising a nucleic acid comprising a nucleotide sequence encoding a PEBL. In some embodiments, the kit comprising a nucleic acid comprising a nucleotide sequence encoding a PEBL, and a nucleic acid comprising a nucleotide sequence encoding a CAR. The kit can be designed according to any of the embodiments described herein.

In certain embodiments, the nucleotide sequence encoding the CAR and/or the nucleotide sequence encoding the PEBL further comprise sequences (e.g., plasmid or vector sequences) that allow, e.g., cloning and/or expression. For example, the nucleotide sequence can be provided as part of a plasmid for ease of cloning into other plasmids and/or expression vectors for, e.g., transfection into a cell (e.g., an immune cell). In certain embodiments, the nucleotide sequence encoding the CAR and the nucleotide sequence encoding the PEBL are provided on a single plasmid or vector. In certain embodiments, the nucleotide sequences are provided on separate plasmids or expression vectors. In some embodiments, the expression vector is selected for viral expression.

Typically, the kits are compartmentalized for ease of use and can include one or more containers with reagents. In certain embodiments, all of the kit components are packaged together. Alternatively, one or more individual components of the kit can be provided in a separate package from the other kits components. The kits can also include instructions for using the kit components.

### VI. Genome Editing

Provided herein is an engineered immune cell containing a genetic modification of a target CD2 nucleic acid sequence. As noted above, downregulation of expression of an immune molecule on an effector T cells can be achieved according to a variety of other known methods including, for example, gene editing methods with meganucleases, TALEN, CRISPR/Cas, and zinc finger nucleases. Thus, in certain embodiments, the engineered immune cell further comprises a modified gene, which modification renders a target gene or protein non-functional. By way of example, the engineered immune cell of the present invention further comprises a modified (e.g., non-functional) CD2 gene (modified using, e.g., meganucleases, TALEN, CRISPR/Cas9, or zinc finger nucleases) that prevents or reduces expression of CD2 protein, and/or otherwise impairs (e.g., structurally) the CD2 protein from being recognized by or interfering with the CAR. Methods of modifying gene expression using such methods are readily available and well-known in the art.

In some embodiments, the CD2 gene in the engineered immune cell is inactivated using a rare-cutting endonuclease specifically catalyzes cleavage in one targeted gene (i.e., CD2) thereby inactivating said targeted gene. The nucleic acid strand breaks caused by the rare-cutting endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Ma, Kim et al. 2003). Repair via non-homologous end joining (NHEJ) often results in small insertions or deletions and can be used for the creation of specific gene knockouts. Said modification may be a substitution, deletion, or addition of at least one nucleotide. Cells in which a cleavage-induced mutagenesis event, i.e a mutagenesis event consecutive to an NHEJ event, has occurred can be identified and/or selected by well-known method in the art.

In particular embodiments, the method for genome editing such an immune cell comprises at least one of the following steps: (1) providing an immune cell, preferably from cell culture or a peripheral blood sample, (b) introducing into said immune cell a rare-cutting endonuclease able to selectively inactivate by DNA cleavage, preferably by double-strand break respectively the CD2 gene in the immune cell; and (c) expanding the resulting immune cell. In some embodiments, the method also includes sorting a population of the resulting immune cells to obtain a substantially pure population of immune cells with an inactivated CD2 gene.

In some embodiments, the CD2 gene in the engineered immune cell is inactivated using a zinc finger nucleases (ZFN) specific for CD2. ZFNs comprise a zinc finger protein that has been engineered to bind to a target site in a gene of choice and cleavage domain or a cleavage half-domain. In some instances, the ZFN comprises a DNA-binding domain that specifically binds to a target site in a human CD2 gene.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties. Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227. ZFNs and methods for design and construction of fusion proteins (and polynucleotides encoding same) are known to those of skill in the art and described in detail in US20050064474 and US20060188987. Additional descriptions of ZFNs can be found in, e.g., US20140030240.

Methods of inactivating a target gene in an immune cell using CRISPR/Cas technology are described, for example, in U.S. Patent Publication Nos. US2016/0272999, US2017/0204372, and US2017/0119820. Zinc finger nuclease systems for inactivating an endogenous gene are described in detail in the specification of U.S. Pat. No. 8,735,153, the disclosure is herein incorporated by reference. The transcription activator-like effector nuclease (TALEN) based genome editing system is described in Gaj et al., Trends in biotechnology, 2013, 31: 397-405, International Patent Publication No. WO2018/073393, and U.S. Patent Publication No. US2016/0120906.

The CRISPR/Cas system is a system for inducing targeted genetic alterations (genome modifications). Target recognition by the Cas9 protein requires a “seed” sequence within the guide RNA (gRNA) and a conserved multinucleotide containing protospacer adjacent motif (PAM) sequence upstream of the gRNA-binding region. The CRISPR/Cas system can thereby be engineered to cleave substantially any DNA sequence by redesigning the gRNA in cell lines, primary cells, and engineered cells. The CRISPR/Cas system can simultaneously target multiple genomic loci by co-expressing a single Cas9 protein with two or more gRNAs, making this system uniquely suited for multiple gene editing or synergistic activation of target genes. Examples of a CRISPR/Cas system used to inhibit gene expression are described in U.S. Publication No.: 2014/0068797 and U.S. Pat. Nos. 8,697,359 and 8,771,945. The system induces permanent gene disruption that utilizes the RNA-guided Cas9 endonuclease to introduce DNA double stranded breaks which trigger error-prone repair pathways to result in frame shift mutations. In some cases, other endonucleases may also be used, including but not limited to, Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, T7, Fok1, other nucleases known in the art, homologs thereof, or modified versions thereof.

CRISPR/Cas gene disruption occurs when a gRNA sequence specific for a target gene and a Cas endonuclease are introduced into a cell and form a complex that enables the Cas endonuclease to introduce a double strand break at the target gene. In some instances, the CRISPR system comprises one or more expression vectors comprising a nucleic acid sequence encoding the Cas endonuclease and a guide nucleic acid sequence specific for the target gene. The guide nucleic acid sequence is specific for a gene and targets that gene for Cas endonuclease-induced double strand breaks. The sequence of the guide nucleic acid sequence may be within a loci of the gene. In some embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40, 45, 50, or more nucleotides in length. The guide nucleic acid sequence includes a RNA sequence, a DNA sequence, a combination thereof (a RNA-DNA combination sequence), or a sequence with synthetic nucleotides, such as a peptide nucleic acid (PNA) or Locked Nucleic Acid (LNA). The guide nucleic acid sequence can be a single molecule or a double molecule. In one embodiment, the guide nucleic acid sequence comprises a single guide RNA. In some embodiments, nucleic acid sequence can be one or more selected from the group consisting of SEQ ID NOS:44-47.

In some embodiments, the engineered immune cell of the present invention can be modified via the CRISPR/Cas system to inactivate the human CD2 gene. Details of the genomic structure and sequence of the human CD2 gene can be found, for example, in NCBI Gene database under Gene ID:914.

Commercially available kits, gRNA vectors and donor vectors, for knockout of specific target genes are available, for example, from Origene (Rockville, Md.), GenScript (Atlanta, Ga.), Applied Biological Materials (ABM; Richmond, British Colombia), BioCat (Heidelberg, Germany) or others. For example, commercially available kits or kit components for knockout of CD2 via CRISPR include, for example, those available as catalog numbers, sc-402105, sc-419537, sc419537, and the like, each available from Santa Cruz Biotechnology.

In some embodiments, any genome editing system can be used to introduce any of the nucleic acid outlined herein into the genome of an immune cell, e.g., a T cell (including but not limited to a gamma-delta T cell) and an NK cell.

Gene silencing of CD2 can be performed according to any method recognized by one skilled in the art. In some cases, a short hairpin RNA that targets CD2 RNA molecules is used. Commercially available reagents for CD2 targeted RNAi include, e.g., those available as catalog number, sc-29970 from Santa Cruz Biotechnology.

### VII. Method of Treating

In one aspect, the present invention relates to the use of an engineered immune cell that comprises a nucleic acid comprising a nucleotide sequence encoding a chimeric antigen receptor (CAR) and a nucleic acid comprising a nucleotide sequence encoding a single-chain variable fragment (scFv) linked to a localizing domain for treating cancer, comprising administering a therapeutic amount of the engineered immune cell to a subject in need thereof. In some embodiments, the cancer is a T cell malignancy. In certain embodiments, the T cell malignancy is early T-cell progenitor acute lymphoblastic leukemia (ETP-ALL). In certain embodiments, the engineered immune cell is administered into the subject by intravenous infusion, intra-arterial infusion, intraperitoneal infusion, direct injection into tumor and/or perfusion of tumor bed after surgery, implantation at a tumor site in an artificial scaffold, or intrathecal administration.

In other aspects, the present invention relates to the use of an engineered immune cell that comprises a nucleic acid comprising a nucleotide sequence encoding a chimeric antigen receptor (CAR), and a nucleic acid comprising a nucleotide sequence encoding a target-binding molecule (e.g., scFv) linked to a localizing domain for treating an autoimmune disorder, comprising administering a therapeutic amount of the engineered immune cell to a subject in need thereof.

In other aspects, the present invention also relates to the use of an engineered immune cell that comprises a nucleic acid comprising a nucleotide sequence encoding a chimeric antigen receptor (CAR), and a nucleic acid comprising a nucleotide sequence encoding a target-binding molecule against CD2 (e.g., anti-CD2 scFv) linked to a localizing domain for treating an infectious disease, comprising administering a therapeutic amount of the engineered immune cell to a subject in need thereof.

In some aspects, the engineered immune cell is administered by infusion into the subject. Methods of infusing immune cells (e.g., allogeneic or autologous immune cells) are known in the art. A sufficient number of cells are administered to the recipient in order to ameliorate the symptoms of the disease. Typically, dosages of 10⁷ to 10¹⁰ cells are infused in a single setting, e.g., dosages of 10⁹ cells. Infusions are administered either as a single 10⁹ cell dose or divided into several 10⁹ cell dosages. The frequency of infusions can be every 3 to 30 days or even longer intervals if desired or indicated. The quantity of infusions is generally at least 1 infusion per subject and preferably at least 3 infusions, as tolerated, or until the disease symptoms have been ameliorated. The cells can be infused intravenously at a rate of 50-250 ml/hr. Other suitable modes of administration include intra-arterial infusion, direct injection into tumor and/or perfusion of tumor bed after surgery, implantation at the tumor site in an artificial scaffold, intrathecal administration, and intraocular administration. Methods of adapting the present invention to such modes of delivery are readily available to one skilled in the art.

In some aspects, provided is a substantially pure population of engineered immune cells comprising any one of the engineered immune cells described herein, wherein at least 90%, e.g., at least 90%, 91%, 92%, 93%, 94%, 95, 96% 97%, 98% 99% or more of the engineered immune cells lack CD2 expression. In some cases, the substantially pure population comprises at least 80%, e.g., at least 80%, 81%, 82%, 83%, 84%, 85%, 86% 87%, 88% 89%, 90%, 91%, 92%, 93%, 94%, 95%, 96% 97%, 98% 99%, or more engineered immune cells lacking CD2 expression.

In other aspects, also provided is a method of treating cancer or an autoimmune disorder in a subject in need thereof, comprising administering a therapeutic amount of an engineered immune cell having any of the embodiments described herein to the subject, thereby treating cancer or an autoimmune disorder in a subject in need thereof. In some aspects, provided is a method of treating cancer or an autoimmune disorder in a subject in need thereof, comprising administering a therapeutic amount of a substantially pure population of engineered immune cells having any of the embodiments described herein to the subject, thereby treating cancer or an autoimmune disorder in a subject in need thereof.

In certain embodiments, the method comprises administering a therapeutic amount of an engineered immune cell comprising a nucleic acid having a nucleotide sequence encoding a target-binding molecule against CD2 linked to a localizing domain, as described herein. In some instances, a second nucleic acid comprises a nucleotide sequence encoding a CAR. In some embodiments, the CAR comprises intracellular signaling domains of 4-1BB and CD3, and an antibody that binds to a cytokine such as CD2.

In some embodiments, the engineered immune cell is autologous to the subject in need of treatment. In other embodiments, the engineered immune cell is allogenic to the subject in need of treatment.

In certain embodiments, the engineered immune cell is administered into the subject by intravenous infusion, intra-arterial infusion, direct injection into tumor and/or perfusion of tumor bed after surgery, implantation at a tumor site in an artificial scaffold, intrathecal administration, and intraocular administration.

In certain embodiments, the engineered immune cell is administered by infusion into the subject. Methods of infusing immune cells (e.g., allogeneic or autologous immune cells) are known in the art. A sufficient number of cells are administered to the recipient in order to ameliorate the symptoms of the disease. Typically, dosages of 10⁷ to 10¹⁰ cells are infused in a single setting, e.g., dosages of 10⁹ cells. Infusions are administered either as a single 10⁹ cell dose or divided into several 10⁹ cell dosages. The frequency of infusions can be daily, every 2 to 30 days or even longer intervals if desired or indicated. The quantity of infusions is generally at least 1 infusion per subject and preferably at least 3 infusions, as tolerated, or until the disease symptoms have been ameliorated. The cells can be infused intravenously at a rate of 50-250 ml/hr. Other suitable modes of administration include intra-arterial infusion, intraperitoneal infusion, direct injection into tumor and/or perfusion of tumor bed after surgery, implantation at the tumor site in an artificial scaffold, intrathecal administration. Methods of adapting the present invention to such modes of delivery are readily available to one skilled in the art.

In certain embodiments, the method of treating cancer according to the present invention is combined with at least one other known cancer therapy, e.g., chemotherapy. In some embodiments, the method of treating cancer according to the present invention is combined therapeutically an agent that suppresses negative checkpoint regulators such an antibody to PD-1, CTLA4, LAG3, TIM3, TIGIT, or another immune checkpoint molecule. This combination may be particularly effective when treating T cell lymphomas, due to immune suppressive environment often present within lymphomas.

In other aspects, also provided is use of an engineered immune cell having any of the embodiments described herein for treating cancer, comprising administering a therapeutic amount of the engineered immune cell to a subject in need thereof.

In certain embodiments, the engineered immune cell is administered into the subject by intravenous infusion, intra-arterial infusion, intraperitoneal infusion, direct injection into tumor and/or perfusion of tumor bed after surgery, implantation at a tumor site in an artificial scaffold, or intrathecal administration.

In some embodiments, the subject is treated with a non-myeloablative chemotherapy prior to an administration (e.g., an infusion) of engineered immune cells outlined herein. In some embodiments, the non-myeloablative chemotherapy is cyclophosphamide 60 mg/kg/d for 2 days (days 27 and 26 prior to infusion of the engineered immune cells) and fludarabine 25 mg/m²/d for 5 days (days 27 to 23 infusion of the engineered immune cells). The subject is administered one or more lymphodepletion (e.g., immunosuppressive conditioning) agents. Non-limiting examples of a preconditioning agent include cyclophosphamide, fludarabine, and any combinations thereof. Detailed methods for conditioning a patient prior to CAR-T cell therapy are found in, for example, U.S. Pat. No. 9,855,298, the contents are incorporated by reference herein in its entireties.

Additional preconditioning methods are described in Gassner et al., Cancer Immunol. Immunother. 2011, 60, 75-85, Muranski et al., Nat. Clin. Pract. Oncol., 2006, 3, 668-681, Dudley, et al., J. Clin. Oncol. 2008, 26, 5233-5239, and Dudley et al., J. Clin. Oncol. 2005, 23, 2346-2357, all of which are incorporated by reference herein in their entireties.

In some embodiments, after receiving non-myeloablative chemotherapy and infusion of the engineered immune cells, the subject receives an intravenous administration of a cytokine, such as IL-2, IL-7, IL-15, or any combination thereof. In some embodiments, after receiving non-myeloablative chemotherapy the patient receives a population of the engineered immune cells in combination with IL-2, IL-7, IL-15, or any combination thereof. In some cases, IL-2, IL-7, IL-15, or any combination thereof are administered after the population of cells. In certain cases, IL-2, IL-7, IL-15, or any combination thereof are administered concomitantly with the population of cells. IL-2 includes IL-2 (aldeskeukin), a biosimilar thereof, or a variant thereof.

In some embodiments, the IL-2 comprises a high-dose IL-2 regimen such as but not limited to, administering intravenously starting on the day after administering a therapeutically effective population of engineered immune cells described herein, wherein the IL-2 is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, the schedule may be repeated for another 14 doses, for a maximum of 28 doses in total.

In other embodiments, IL-2 is administered intravenously at a dose of about 18×10⁶ IU/m² over 6 hours, followed by a dose of 18×10⁶ IU/m² over 12 hours, followed by a dose of 18×10⁶ IU/m² over 24 hours, and followed by a dose of 18×10⁶ IU/m² over 72 hours. Such a treatment regimen can be repeated every 28 days for a maximum of four cycles. In some embodiments, the IL-2 regimen comprises 18,000,000 IU/m² on day 1, and 9,000,000 IU/m² on day 2, and 4,500,000 IU/m² on days 3 and 4. In another embodiment, the IL-2 regimen comprises administration of pegylated IL-2 every 1, 2, 4, 6, 7, 14 or 21 days at a dose of 0.10 mg/day to 50 mg/day.

In some embodiments, the engineered immune cells or the population of the engineered immune cells are administered as part of a combination treatment, such as simultaneously with or sequentially with, in any order, another therapeutic intervention, such as an antibody or engineered cell or receptor or agent, such as a cytotoxic or therapeutic agent. In some embodiments, the cells are co-administered with one or more additional therapeutic agents or in connection with another therapeutic intervention, either simultaneously or sequentially in any order. In some embodiments, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent. In some embodiments, the therapeutic agent suppresses negative checkpoint regulators, such as but not limited to an antibody to PD-1, CTLA4, LAG3, TIM3, TIGIT, or another immune checkpoint molecule.

Following administration of the engineered immune cells described herein, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in cancer burden or load.

### VIII. Exemplary Embodiments of the Invention

In one aspect, the invention provides a polynucleotide encoding an anti-CD2-4-1BB-CD3 chimeric antigen receptor (CAR) comprising an anti-CD2 single chain variable fragment (scFv) domain, a CD8α hinge-transmembrane domain, a 4-1BB intracellular signaling domain, and a CD3ζ signaling domain.

Of the polynucleotide of any embodiment, said anti-CD2 scFv domain of the CAR comprises an amino acid sequence having at least 90% (e.g., 90%, 91%, 92%, 93%, 94%, 95%, 96%, 97%, 98%, 99%, or 100%) sequence identity of SEQ ID NO:22 or SEQ ID NO:23. Of the polynucleotide of any embodiment, said CD8α hinge-transmembrane domain of the CAR comprises an amino acid sequence having at least 90% (e.g., 90%, 91%, 92%, 93%, 94%, 95%, 96%, 97%, 98%, 99%, or 100%) sequence identity of SEQ ID NO:15. Of the polynucleotide of any embodiment, said 4-1BB intracellular signaling domain of the CAR comprises an amino acid sequence having at least 90% (e.g., 90%, 91%, 92%, 93%, 94%, 95%, 96%, 97%, 98%, 99%, or 100%) sequence identity of SEQ ID NO:16. Of the polynucleotide of any embodiment, said CD3ζ signaling domain of the CAR comprises an amino acid sequence having at least 90% sequence identity of SEQ ID NO:17. Of the polynucleotide of any embodiment, the CAR comprises an amino acid sequence having at least 90% (e.g., 90%, 91%, 92%, 93%, 94%, 95%, 96%, 97%, 98%, 99%, or 100%) sequence identity to SEQ ID NO:5. In some cases, the CAR has a nucleic acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NO:10.

Provided herein is an isolated viral vector comprising any one of the polynucleotides encoding the CAR described herein. In some aspects of the invention, the isolated viral vector comprising any one of the polynucleotides encoding the CAR outlined herein is introduced into an immune cell.

Also provided herein is an engineered immune cell comprising the anti-CD2-4-1BB-CD3 chimeric antigen receptor described herein. The engineered immune cell of any embodiment is an engineered allogeneic cell. The engineered immune cell of any embodiment is an engineered autologous cell. The engineered immune cell of any embodiment is an engineered T cell. The engineered immune cell of any embodiment is an engineered NK cell.

Provided herein an isolated viral vector comprising a polynucleotide encoding a CD2 blocking polypeptide comprising a single chain variable fragment (scFv) that binds CD2 linked to the N-terminus of a cellular localizing domain, wherein cellular localizing domain comprises an amino acid sequence selected from the group consisting of an endoplasmic reticulum (ER) retention sequence, a Golgi retention sequence, and a proteosome localizing sequence, and wherein said CD2 blocking polypeptide binds endogenous CD2 within a cell.

Of the isolated viral vector of any one of the embodiments, said scFv comprises: (i) a variable heavy chain (VH) sequence having at least 90% (e.g., 90%, 91%, 92%, 93%, 94%, 95%, 96%, 97%, 98%, 99%, or 100%) sequence identity to SEQ ID NO:18 and a variable light chain (VL) sequence having at least 90% (e.g., 90%, 91%, 92%, 93%, 94%, 95%, 96%, 97%, 98%, 99%, or 100%) sequence identity to SEQ ID NO:19, or (ii) variable heavy chain (VH) sequence having at least 90% (e.g., 90%, 91%, 92%, 93%, 94%, 95%, 96%, 97%, 98%, 99%, or 100%) sequence identity to SEQ ID NO:20 and a variable light chain (VL) sequence having at least 90% (e.g., 90%, 91%, 92%, 93%, 94%, 95%, 96%, 97%, 98%, 99%, or 100%) sequence identity to SEQ ID NO:21.

Of the isolated viral vector of any one of the embodiments, said ER retention sequence comprises an amino acid sequence selected from the group consisting of KDEL, KKXX, KKMP, and KKTN, wherein X can be any amino acid; or said Golgi retention sequence is selected from the group consisting of YGRL (SEQ ID NO:40), YQRL (SEQ ID NO:41), YKGL (SEQ ID NO:42), and YXXL (SEQ ID NO:43), wherein X can be any amino acid. Of the isolated viral vector of any one of the embodiments, said CD2 blocking polypeptide further comprises a transmembrane domain linked between said scFv and said ER retention sequence domain comprising KKMP or KKTN or said Golgi retention sequence domain comprising YGRL, YQRL, YKGL, wherein said transmembrane domain is a transmembrane domain selected from any one of CD8 α, CD8β, 4-IBB, CD28, CD34, CD4, FcεRIγ, CD16, OX40, CD3ζ, CD3ε, CD3γ, CD3δ, TCRα, CD32, CD64, VEGFR2, FAS, and FGFR2B. Of the isolated viral vector of any one of the embodiments, said transmembrane domain comprises a hinge-transmembrane domain of CD8α.

Of the isolated viral vector of any one of the embodiments, said CD2 blocking polypeptide comprises an amino acid sequence having at least 90% (e.g., 90%, 91%, 92%, 93%, 94%, 95%, 96%, 97%, 98%, 99%, or 100%) sequence identity to any one selected from the group consisting of SEQ ID NOS:1-4. In some embodiments, the CD2 blocking polypeptide comprises a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs:6-9.

In some aspects of the invention, the isolated viral vector comprising a polynucleotide encoding any of CD2 blocking polypeptides outlined herein is introduced into an immune cell.

Also provided herein is an engineered immune cell comprising the CD2 blocking polypeptides described herein. The engineered immune cell of any embodiment is an engineered allogeneic cell. The engineered immune cell of any embodiment is an engineered autologous cell. The engineered immune cell of any embodiment is an engineered T cell. The engineered immune cell of any embodiment is an engineered NK cell.

In one aspect, provided herein is an engineered immune cell comprising a polypeptide comprising a target-binding molecule linked to a cellular localizing domain, wherein the target-binding molecule is an antibody that binds CD2 protein (e.g., human CD2 protein), the cellular localizing domain comprises an amino acid sequence selected from the group consisting of an endoplasmic reticulum (ER) retention sequence, a Golgi retention sequence, and a proteosome localizing sequence, and the target-binding molecule linked to the localizing domain is not secreted by the engineered cell.

In some embodiments, the antibody that binds the CD2 protein (e.g., human CD2 protein) is an anti-CD2 single chain variable fragment (scFv). In some embodiments, the scFv comprises a variable heavy chain (V_{H}) sequence having at least 90% sequence identity to SEQ ID NO:18 and a variable light chain (V_{L}) sequence having at least 90% sequence identity to SEQ ID NO:19. In some embodiments, the scFv comprises a variable heavy chain (V_{H}) sequence set forth in SEQ ID NO:18 and a variable light chain (V_{L}) sequence set forth in SEQ ID NO:19.

In some embodiments, the scFv comprises a variable heavy chain (V_{H}) sequence having at least 90% sequence identity to SEQ ID NO:20 and a variable light chain (V_{L}) sequence having at least 90% sequence identity to SEQ ID NO:21. In certain embodiments, the scFv comprises a variable heavy chain (V_{H}) sequence set forth in SEQ ID NO:20 and a variable light chain (V_{L}) sequence set forth in SEQ ID NO:21.

In some embodiments, the cellular localizing domain comprises an amino acid sequence selected from KDEL, KKXX, KKMP, or KKTN, wherein X can be any amino acid. In certain embodiments, the polypeptide further comprises a transmembrane domain linked between the target-binding molecule and the cellular localizing domain. In some case, the transmembrane domain is derived from CD8α, CD8β, 4-IBB, CD28, CD34, CD4, FcεRIγ, CD16, OX40, CD3ζ, CD3ε, CD3γ, CD3δ, TCRα, CD32, CD64, VEGFR2, FAS, or FGFR2B.

In certain embodiments, the transmembrane domain comprises a hinge-transmembrane domain derived from CD8α.

In various embodiments, the polypeptide of the engineered immune cell comprises an amino acid sequence having at least 90% (e.g., 90%, 91%, 92%, 93%, 94%, 95%, 96%, 97%, 98%, 99%, or 100%) sequence identity to SEQ ID NO:1 or SEQ ID NO:3. The polypeptide may comprise an amino acid sequence of SEQ ID NO:1 or SEQ ID NO:3.

In certain embodiments, the polypeptide of the engineered immune cell comprises an amino acid sequence having at least 90% (e.g., 90%, 91%, 92%, 93%, 94%, 95%, 96%, 97%, 98%, 99%, or 100%) sequence identity to SEQ ID NO:2 or SEQ ID NO:4. The polypeptide may comprise an amino acid sequence of SEQ ID NO:2 or SEQ ID NO:4.

In some embodiments, the engineered immune cell further comprises a chimeric antigen receptor (CAR). In certain instances, the CAR is an anti-CD2-4-1BB-CD3ζ CAR. The anti-CD2-4-1BB-CD3ζ CAR may comprise an amino acid sequence having at least 90% sequence identity to SEQ ID NO:5. The anti-CD2-4-1BB-CD3ζ CAR can bind CD2 (e.g., human CD2). In some instances, such an anti-CD2-4-1BB-CD3ζ CAR can be referred to as a “CD2 CAR”.

In some embodiments, the engineered immune cell induces cytotoxicity of CD2+ cells.

In some embodiments, endogenous CD2 expression is blocked in the engineered immune cell. The blockage of endogenous CD2 expression may persist for at least 6 months. The blockage of endogenous CD2 expression may persist for at least 12 months.

In some embodiments, the engineered immune cell proliferates at a substantially equivalent rate as a comparable immune cell. In some embodiments, the engineered immune cell is an engineered allogeneic cell or an engineered autologous cell. In other embodiments, the engineered immune cell is an engineered T cell such as a gamma-delta T cell.

In another aspect, provided herein is a method of treating cancer or an autoimmune disease in a subject in need thereof comprising administering a therapeutic amount of a composition comprising any one of the engineered immune cell described herein to the subject, thereby treating cancer or the autoimmune disease in a subject in need thereof. In some instances, the composition further comprises a pharmaceutically acceptable carrier. The cancer may be a T cell malignancy or a CD2 associated cancer. In one embodiment, the T cell malignancy is early T cell progenitor acute lymphoblastic leukemia (ETP-ALL) or another T cell leukemia. In another embodiment the T cell malignancy is a lymphoma, including but not limited to, Cutaneous T-Cell Lymphoma (CTCL), Mycosis Fungoides, Sezary Syndrome or Peripheral T cell Lymphoma (PTCL).

In some embodiments, the administration is by intravenous infusion, intra-arterial infusion, intraperitoneal infusion, direct injection into tumor and/or perfusion of tumor bed after surgery, implantation at a tumor site in an artificial scaffold, or intrathecal administration.

In another aspect, provided herein is a polynucleotide encoding a polypeptide comprising a target-binding molecule linked to a cellular localizing domain. In some cases, the target-binding molecule is an antibody that binds CD2 protein (e.g., human CD2 protein), the cellular localizing domain comprises an amino acid sequence selected from the group consisting of an endoplasmic reticulum (ER) retention sequence, a Golgi retention sequence, and a proteosome localizing sequence, and the target-binding molecule linked to the localizing domain is not secreted by the engineered cell.

In particular embodiments, the antibody that binds the CD2 protein (e.g., human CD2 protein) is an anti-CD2 single chain variable fragment (scFv). In certain embodiments, the scFv comprises a variable heavy chain (V_{H}) sequence having at least 90% (e.g., 90%, 91%, 92%, 93%, 94%, 95%, 96%, 97%, 98%, 99%, or 100%) sequence identity to SEQ ID NO:18 and a variable light chain (V_{L}) sequence having at least 90% (e.g., 90%, 91%, 92%, 93%, 94%, 95%, 96%, 97%, 98%, 99%, or 100%) sequence identity to SEQ ID NO:19. In some embodiments, the scFv comprises a variable heavy chain (V_{H}) sequence set forth in SEQ ID NO:18 and a variable light chain (V_{L}) sequence set forth in SEQ ID NO:19.

In other embodiments, the scFv comprises a variable heavy chain (V_{H}) sequence having at least 90% (e.g., 90%, 91%, 92%, 93%, 94%, 95%, 96%, 97%, 98%, 99%, or 100%) sequence identity to SEQ ID NO:20 and a variable light chain (V_{L}) sequence having at least 90% (e.g., 90%, 91%, 92%, 93%, 94%, 95%, 96%, 97%, 98%, 99%, or 100%) sequence identity to SEQ ID NO:21. In certain embodiments, the scFv comprises a variable heavy chain (V_{H}) sequence set forth in SEQ ID NO:20 and a variable light chain (V_{L}) sequence set forth in SEQ ID NO:21.

In some embodiments, the cellular localizing domain comprises an amino acid sequence selected from KDEL, KKXX, KKMP, or KKTN, wherein X can be any amino acid. In certain embodiments, the polypeptide further comprises a transmembrane domain linked between the target-binding molecule and the cellular localizing domain. In some cases, the transmembrane domain is derived from CD8α, CD8β, 4-IBB, CD28, CD34, CD4, FcεRIγ, CD16, OX40, CD3ζ, CD3ε, CD3γ, CD3δ, TCRα, CD32, CD64, VEGFR2, FAS, or FGFR2B.

In certain embodiments, the transmembrane domain comprises a hinge-transmembrane domain derived from CD8α.

In certain embodiments, the polypeptide of the engineered immune cell comprises an amino acid sequence having at least 90% (e.g., 90%, 91%, 92%, 93%, 94%, 95%, 96%, 97%, 98%, 99%, or 100%) sequence identity to SEQ ID NO:1 or SEQ ID NO:3. The polypeptide may comprise an amino acid sequence of SEQ ID NO:1 or SEQ ID NO:3.

In various embodiments, the polypeptide of the engineered immune cell comprises an amino acid sequence having at least 90% (e.g., 90%, 91%, 92%, 93%, 94%, 95%, 96%, 97%, 98%, 99%, or 100%) sequence identity to SEQ ID NO:2 or SEQ ID NO:4. The polypeptide may comprise an amino acid sequence of SEQ ID NO:2 or SEQ ID NO:4.

In certain embodiments, the PEBL described herein comprises a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs:6-9.

In some embodiments, provided herein is an expression vector comprising any one of polynucleotide described herein. In some cases, the expression vector also includes a nucleic acid sequence encoding a chimeric antigen receptor (CAR). The CAR can be an anti-CD2-4-1BB-CD3ζ CAR. In some embodiments, the anti-CD2-4-1BB-CD3ζ CAR comprises an amino acid sequence having at least 90% (e.g., 90%, 91%, 92%, 93%, 94%, 95%, 96%, 97%, 98%, 99%, or 100%) sequence identity to SEQ ID NO:5.

In some embodiments, the expression vector comprises a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NO:10. In some cases, the expression vector comprises the nucleic acid sequence of SEQ ID NO:10. In some embodiments, the expression vector comprises a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs:6-9. In some cases, the expression vector comprises the nucleic acid sequence of any one of SEQ ID NOS:6-9.

In some embodiments, provided herein a host cell comprising any one of the expression vectors described herein.

In yet another aspect, provided herein is a method for producing an engineered immune cell, the method comprising: introducing into an immune cell any one of the polynucleotides or expression vectors disclosed herein. In some embodiments, the endogenous CD2 expression is blocked in the engineered immune cell. In some embodiments, the engineered immune cell is an engineered allogeneic cell or an engineered autologous cell. In other embodiments, the engineered immune cell is an engineered T cell such as a gamma-delta T cell.

In one aspect, provided herein is an isolated anti-CD2-4-1BB-CD3ζ chimeric antigen receptor (CAR) molecule comprising an anti-CD2 single chain variable fragment (scFv) domain, a CD8α hinge-transmembrane domain, a 4-1BB intracellular signaling domain, and a CD3t signaling domain. The anti-CD2 single chain variable fragment (scFv) domain can comprise an amino acid sequence having at least 90% (e.g., 90%, 91%, 92%, 93%, 94%, 95%, 96%, 97%, 98%, 99%, or 100%) sequence identity of SEQ ID NO:22 or SEQ ID NO:23. The CD8α hinge-transmembrane domain can include an amino acid sequence having at least 90% sequence identity of SEQ ID NO:15. The 4-1BB intracellular signaling domain may contain an amino acid sequence having at least 90% (e.g., 90%, 91%, 92%, 93%, 94%, 95%, 96%, 97%, 98%, 99%, or 100%) sequence identity of SEQ ID NO:16. The CD3ζ signaling domain may contain an amino acid sequence having at least 90% (e.g., 90%, 91%, 92%, 93%, 94%, 95%, 96%, 97%, 98%, 99%, or 100%) sequence identity of SEQ ID NO:17. In some embodiments, the isolated CAR molecule comprises an amino acid sequence having at least 90% (e.g., 90%, 91%, 92%, 93%, 94%, 95%, 96%, 97%, 98%, 99%, or 100%) sequence identity to SEQ ID NO:5. In another aspect, provided herein is an isolated nucleic acid molecule encoding any one of the isolated CAR molecules described herein. The anti-CD2-4-1BB-CD3ζ CAR can bind CD2 (e.g., human CD2).

In another aspect, provided herein is an engineered immune cell comprising a polypeptide comprising an anti-CD2-4-1BB-CD3ζ chimeric antigen receptor (CAR) comprising an anti-CD2 single chain variable fragment (scFv) domain, a CD8α hinge-transmembrane domain, a 4-1BB intracellular signaling domain, and a CD3ζ signaling domain. The anti-CD2-4-1BB-CD3ζ CAR can bind CD2 (e.g., human CD2).

In some embodiments, the anti-CD2 single chain variable fragment (scFv) domain comprises an amino acid sequence having at least 90% sequence identity of SEQ ID NO:22 or SEQ ID NO:23. In several embodiments, the CD8α hinge-transmembrane domain includes an amino acid sequence having at least 90% (e.g., 90%, 91%, 92%, 93%, 94%, 95%, 96%, 97%, 98%, 99%, or 100%) sequence identity of SEQ ID NO:15. In certain embodiments, the 4-1BB intracellular signaling domain contains an amino acid sequence having at least 90% (e.g., 90%, 91%, 92%, 93%, 94%, 95%, 96%, 97%, 98%, 99%, or 100%) sequence identity of SEQ ID NO:16. In particular embodiments, the CD3ζ signaling domain comprises an amino acid sequence having at least 90% sequence identity of SEQ ID NO:17. In some embodiments, the isolated CAR molecule comprises an amino acid sequence having at least 90% (e.g., 90%, 91%, 92%, 93%, 94%, 95%, 96%, 97%, 98%, 99%, or 100%) sequence identity to SEQ ID NO:5. In some embodiments, the isolated CAR molecule comprises the amino acid sequence of SEQ ID NO:5. In some embodiments, the isolated CAR molecule comprises the nucleic acid sequence of SEQ ID NO:10.

In some embodiments, the engineered immune cell is an engineered allogeneic cell or an engineered autologous cell. In other embodiments, the engineered immune cell is an engineered T cell such as a gamma-delta T cell.

The contents such as the specification, claims, and figures of WO 2016/126213 and PCT/US2017/063048 are incorporated herein by reference in its entirety for all purposes.

### EXAMPLES

### Example 1: Blockade of CD2 Expression in T Cells for Effective Chimeric Antigen Receptor Therapies

This examples illustrates blockade of CD2 expression with a novel method, combined with a second-generation CAR, resulting in potent anti-CD2 CAR-T cells. This practical strategy provides a new treatment option for patients with cancer.

FIG. 1 provides an exemplary anti-CD2 chimeric antigen receptor (CAR). The scFv of the anti-CD2 monoclonal antibody 9.6 was joined to the CD8α signal peptide, CD8α hinge-transmembrane domain, and the intracellular domains of 4-1BB and CD3ζ of the anti-CD19-4-1BB-CD3 CAR previously developed in the laboratory. The scFv of the anti-CD2 monoclonal antibody 9.6 or 9-1 was joined to the CD8α signal peptide, and a sequence encoding a localizing domain, and optionally, a CD8α hinge-transmembrane domain. These were subcloned into a murine stem cell virus (MSCV) vector. In some cases, the MSCV is a MSCV-internal ribosome entry site (IRES)-green fluorescent protein (GFP) retroviral vector containing a firefly luciferase gene.

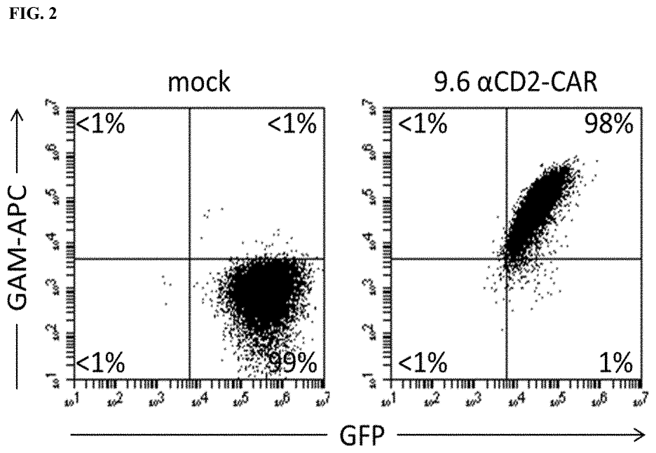

The 9.6 anti-CD2 CAR retroviral vector construct was transduced into Jurkat cells (a leukemia cell line). Preparation of retroviral supernatant and transduction were performed according to standard protocols known to those skilled in the art. The expression results are shown in FIG. 2.

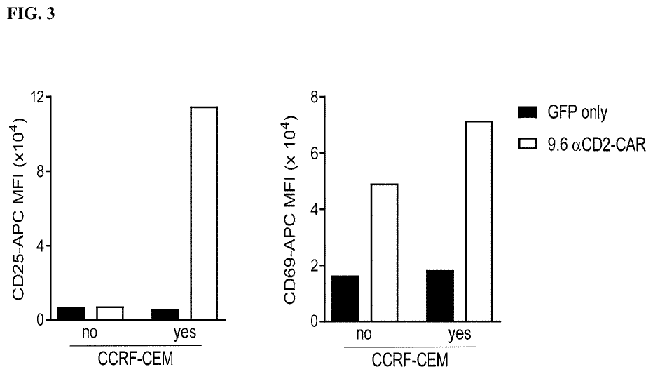

CCRF-CEM cells with the CD2 gene were also transduced with the 9.6 anti-CD2 CAR retroviral vector construct. The resulting cells were maintained in RPMI-1640 media supplemented with 10% FBS and 1% Pen-Strep. The activity of the 9.6 anti-CD2 was assessed. FIG. 3 shows that the anti-CD2 CAR induced expression of CD2 and CD69 (activation markers) in the presence of CD2 target cells.

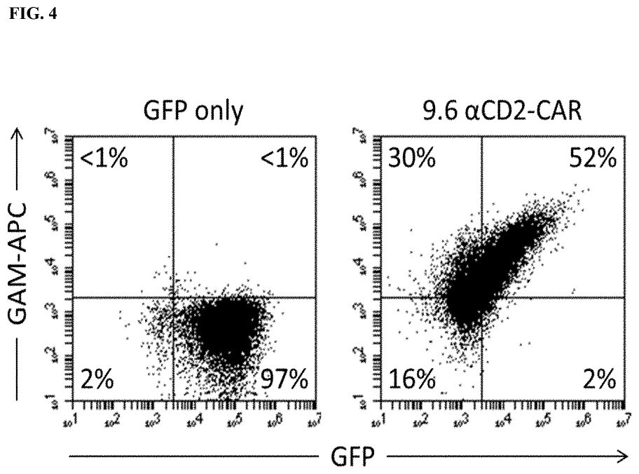

To determine the effect of the anti-CD2 CAR in peripheral blood T lymphocytes, the anti-CD2 CAR was introduced into primary T cells by retroviral transduction or electroporation. FIG. 4 shows the CAR expression.

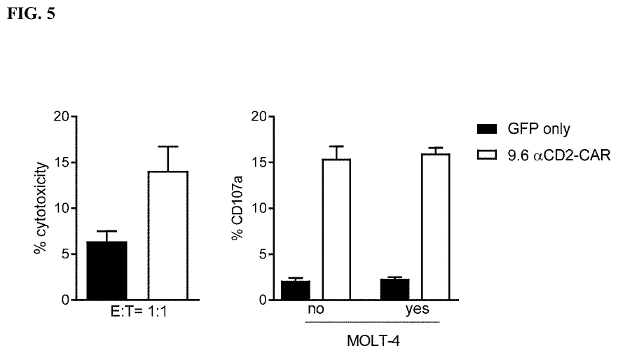

FIG. 5 shows the function of the anti-CD2 CAR when CD2+ target cells (MOLT-4) were cocultured with Jurkat cells transduced with the anti-CD2 CAR or transduced with a vector containing GFP only. In some cases, the cells were co-cultured at 1:1 E:T. The results show that the 9.6 anti-CD2 CAR-T cells exert cytotoxicity against CD2+ target cells.

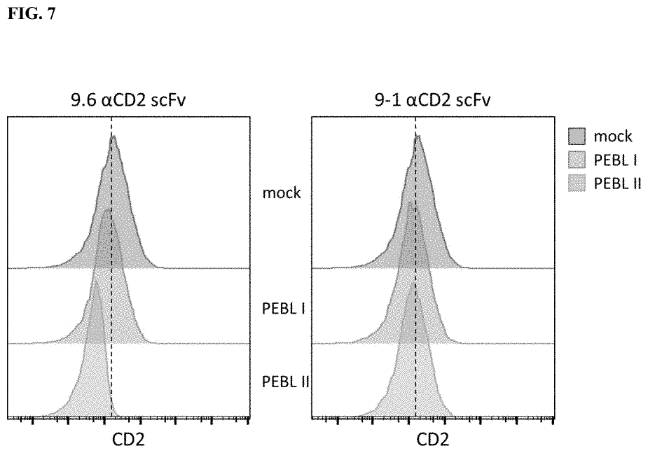

FIG. 6 shows exemplary embodiments of anti-CD2 PEBL constructs. 9.6 PEBLs and 9-1 PEBLs were retrovirally transduced into Jurkat cells. The histograms of FIG. 7 show downregulation of CD2 expression.

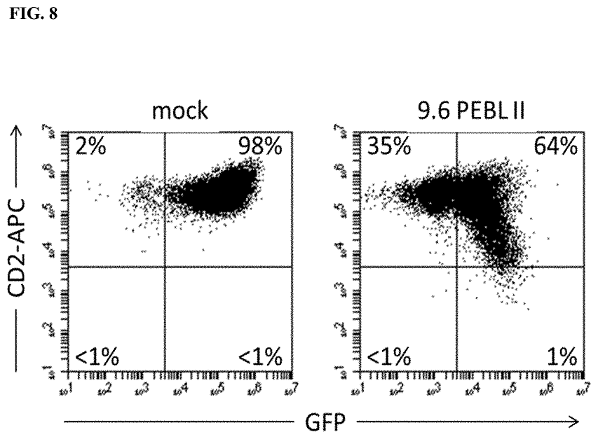

FIG. 8 shows that the 9.6 PEBL II downregulates CD2 expression in human peripheral blood T cells.

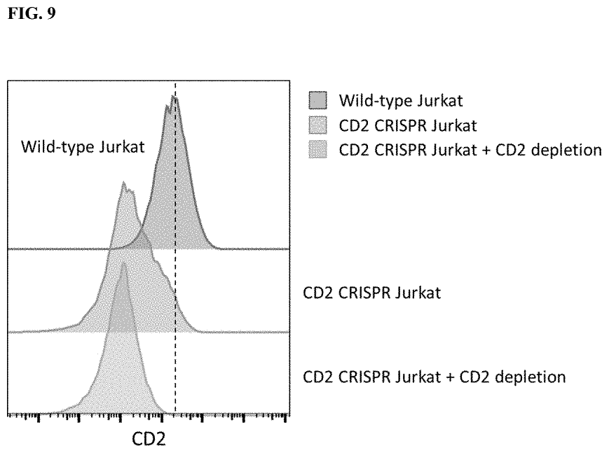

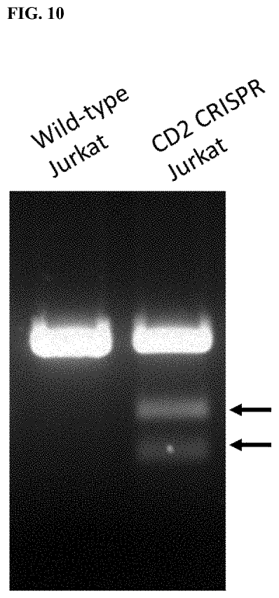

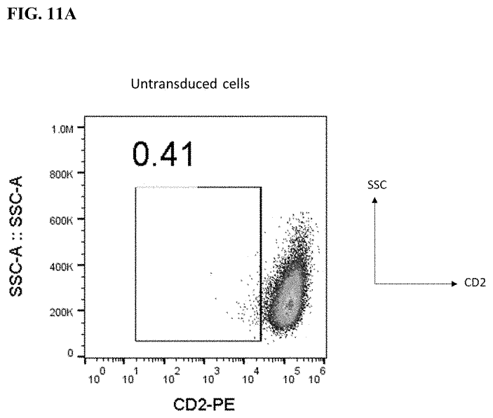

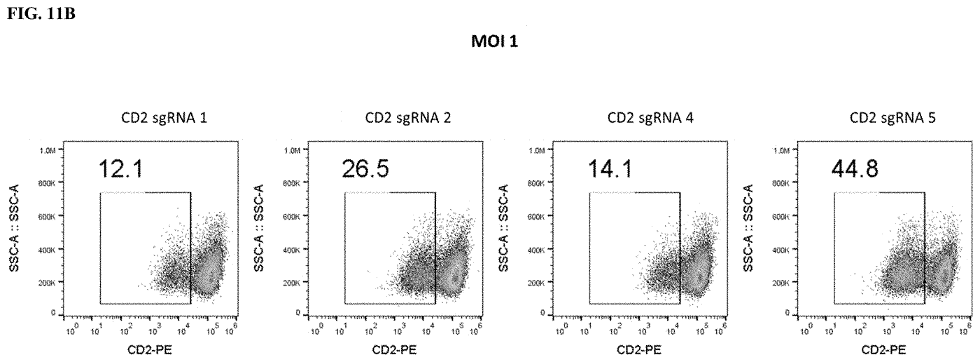

Jurkat cells underwent CRISPR/Cas9 genome editing to inactivate endogenous CD2 expression. In addition, the gene edited Jurkat cells were transduced with an anti-CD2 PEBL. The histogram of FIG. 9 shows downregulation of CD2 in the Jurkat cells. FIG. 10 shows cleavage products in the gene edited Jurkat cells.

### Example 2: Genetic Modification of CD2 in Jurkat Cells

This example illustrates the downregulation of endogenous CD2 expression in Jurkat cells using a CRISPR-based platform.

These gRNA sequences were used with WT SpCas9 to introduce a DSB for genome editing. These sgRNA sequences were described and validated in Sanjana et al., Nat Methods, 2014 August, 11(8):783-4.

TABLE 2
Exemplary guide RNA target sequences.

| SEQ ID NO: | gRNA Name | gRNA Target Sequence |
|---|---|---|
| SEQ ID NO: 44 | CD2 CRISPR guide | CAAAGAGATTACGAATGCCT |
| | RNA 1 | |
| SEQ ID NO: 45 | CD2 CRISPR guide | CTTGTAGATATCCTGATCAT |
| | RNA 2 | |
| SEQ ID NO: 46 | CD2 CRISPR guide | CACGCACCTGGACAGCTGAC |
| | RNA 4 | |
| SEQ ID NO: 47 | CD2 CRISPR guide | GGTTGTGTTGATACAAGTCC |
| | RNA 5 | |

### Lentivirus Constructs

All four CD2 CRISPR sgRNA constructs were cloned into the pLentiCRISPR v2 lentiviral vector by Genscript.

### Cell Culture

293T cells (ATCC CRL-3216) were maintained in DMEM (Gibco) with 10% FBS (Hyclone), 100U/mL penicillin and 100 ug/mL streptomycin (Gibco). Jurkat-E6 cells were maintained in RPMI1640 (Gibco) with 10% FBS (Hyclone), 100U/mL penicillin, 100 ug/mL streptomycin (Gibco) and 1× GlutaMAX (Gibco).

### Virus Production

293T cells were cotransfected with lentiviral transfer vectors and Virapower packaging plasmids mix (Invitrogen) at a 1:4 ratio of DNA:Lipofectamine 2000 (Invitrogen). Transfection medium was replaced with fresh DMEM (Gibco) with 10% FBS (Hyclone) 6 hours post transfection. 3 days later, the virus supernatant was collected, passed through a 0.45 μM filter and then concentrated 100× using Lenti-X Concentrator (Clontech). Concentrated virus stock was stored at −140° C. until use.

### Virus Titration on 293T Cells

293T cells were transduced with varying volumes of lentiviruses in the presence of 5 μg/mL polybrene (Sigma). After 15h overnight culture, transduction medium was removed and cells were treated with 10U/mL DNaseI (New England Biolabs) in fresh culture media for 1 hour at 37° C. The media was then replaced with fresh DMEM with 10% FBS for further culture. Transduced cells were harvested for analysis at >72h post transduction. Virus titers were determined using RT-qPCR.

Integration unit (IU) titers were calculated from RT-qPCR data on genomic DNA using the equation: [IU/ml=(Number of 293T cells per sample x number of proviral gene copies per genome) Virus volume in mL]. IU titers were calculated using samples within the linear range of proviral gene copy number and virus volume.

### Jurkat Transduction

Jurkat cells were transduced with lentiviral vectors at MOI 1 or 5. Transduced cells were harvested for analyses at >72h post transduction.

### Flow Cytometry

Antibody staining and washes are performed with staining buffer (1×PBS pH7.4, 0.2% BSA, 0.02% sodium azide). Cells were incubated with antibodies on ice for 20 min and washed 3 times. The following antibodies were used for staining: anti-human CD2-PE (clone RPA-2.10, eBioscience 12-0029-42), anti-human CD3 eFluor780 (clone OKT3, eBioscience 47-0037-42). DAPI was used at 2 μg/mL for live/dead discrimination. Stained cells were collected on an Invitrogen Attune NxT flow cytometer and analyzed with FlowJo v10 software.

### Real-Time Quantitative PCR (RT-qPCR)

Genomic DNA was extracted from cells using the DNeasy Blood and Tissue Kit (Qiagen) and RNase A (Qiagen). All kits were used according to manufacturer's recommendations. RT-qPCR was performed using iTaq Universal SYBR Green Supermix (Bio-Rad) on a CFX96 Touch™ Real-Time PCR Detection System (Bio-Rad).

Primers used were: RPPH1-F: 5′-AGCTTGGAACAGACTCACGG-3′ (SEQ ID NO:48); RPPH1-R: 5′-AATGGGCGGAGGAGAGTAGT-3′ (SEQ ID NO:49); WPRE-F: 5′-CCTTTCCGGGACTTTCGCTTT-3′ (SEQ ID NO:50); WPRE-R: 5′-GCAGAATCCAGGTGGCAACA-3′ (SEQ ID NO:51); VSVG-F: 5′-AGGGAACTGTGGGATGACTG-3′ (SEQ ID NO:52); and VSVG-R: 5′-GAACACCTGAGCCTTTGAGC-3′ (SEQ ID NO:53). All assay primers had primer efficiencies between 90% and 110%.

Fold changes of all genes were normalized to a housekeeping gene using the equation: [Fold change=2\hat{ }−(Ct^{(target gene)}−Ct^{(housekeeping gene)}]. Copy number of target genes was normalized to the genomic copy number of RNaseP.

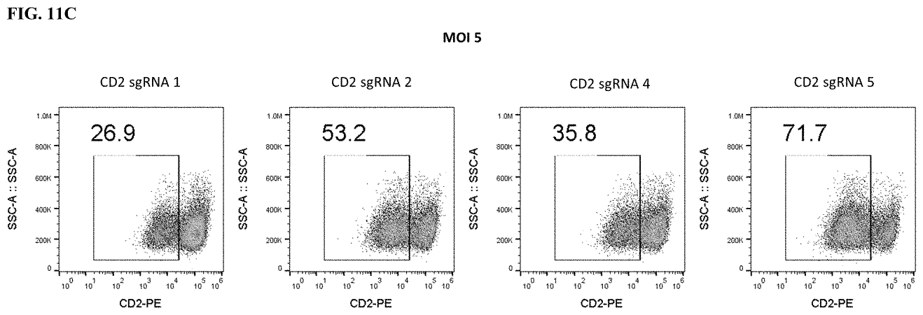

FIG. 11 shows expression of CD2 on transduced Jurkat cells as determined by flow cytometry. Jurkat cells were transduced with the indicated lentiviruses at MOI 1 or 5 and analysed by flow cytometry for CD2 expression 8 days after transduction.

12.2% Jurkat cells transduced with lentiviruses carrying CD2 sgRNA1 at MOI 1 exhibited CD2 knockdown. 26.9% Jurkat cells transduced with the lentiviruses at MOI 5 exhibited CD2 knockdown.

26.5% Jurkat cells transduced with lentiviruses carrying CD2 sgRNA2 at MOI 1 exhibited CD2 knockdown. 53.2% Jurkat cells transduced with the lentiviruses at MOI 5 exhibited CD2 knockdown.

14.1% Jurkat cells transduced with lentiviruses carrying CD2 sgRNA4 at MOI 1 exhibited CD2 knockdown. 35.8% Jurkat cells transduced with the lentiviruses at MOI 5 exhibited CD2 knockdown.

44.8% Jurkat cells transduced with lentiviruses carrying CD2 sgRNA5 at MOI 1 exhibited CD2 knockdown. 71.7% Jurkat cells transduced with the lentiviruses at MOI 5 exhibited CD2 knockdown.

The data shows effective reduction or decreased CD2 expression (e.g., cell surface expression) in Jurkat cells that have undergone CRISPR/Cas9 based gene modification.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. An engineered immune cell comprising:
(i) a chimeric antigen receptor (CAR) comprising a CD2 targeting domain, a transmembrane domain, and an intracellular signaling domain; and
(ii) a genetic modification of a target CD2 nucleic acid sequence to downregulate endogenous CD2 expression in said cell.

2. The engineered immune cell of claim 1, wherein said genetic modification comprises genome editing using a system selected from the group consisting of CRISPR/Cas, zinc finger nucleases, TALENs, and meganucleases.

3. (canceled)

4. The engineered immune cell of claim 2, wherein the CRISPR/Cas system comprises a guide RNA corresponding to a nucleic acid sequence selected from the group consisting of SEQ ID NOS:44-47.

5. The engineered immune cell of claim 1, wherein said CD2 targeting domain comprises a scFv comprising a variable heavy chain (V_{H}) sequence having at least 90% sequence identity to SEQ ID NO:18 and a variable light chain (V_{L}) sequence having at least 90% sequence identity to SEQ ID NO:19.

6. The engineered immune cell of claim 1, wherein said CD2 targeting domain comprises a scFv comprising variable heavy chain (V_{H}) sequence having at least 90% sequence identity to SEQ ID NO:20 and a variable light chain (V_{L}) sequence having at least 90% sequence identity to SEQ ID NO:21.

7. The engineered immune cell of claim 1, wherein said transmembrane domain comprises a hinge-transmembrane domain of CD8α.

8. The engineered immune cell of claim 1, wherein said intracellular signaling domain comprises one or more selected from the group consisting of a 4-1BB signaling domain, a CD28 signaling domain, an OX40 signaling domain, and a CD3ζ signaling domain.

9. The engineered immune cell of claim 1, wherein said CAR is an anti-CD2-4-1BB-CD3ζ CAR comprising an amino acid sequence having at least 90% sequence identity to SEQ ID NO:5.

10. The engineered immune cell of claim 1, wherein said engineered immune cell induces cytotoxicity of CD2+ cells.

11. (canceled)

12. The engineered immune cell of claim 1, wherein said engineered immune cell is an allogeneic cell.

13. The engineered immune cell of claim 1, wherein said engineered immune cell is an autologous cell.

14. The engineered immune cell of claim 1, wherein said engineered immune cell is selected from the group consisting of an engineered T cell, an engineered gamma-delta T cell, and an engineered NK cell.

15.-16. (canceled)

17. The engineered immune cell of claim 1, further comprising a blocking polypeptide comprising a single chain variable fragment (scFv) linked to the N-terminus of a cellular localizing domain,
wherein said scFv binds to a cell surface molecule, wherein said cellular localizing domain comprises an amino acid sequence selected from the group consisting of an endoplasmic reticulum (ER) retention sequence, a Golgi retention sequence, and a proteosome localizing sequence, and wherein said blocking polypeptide remains intracellularly within said engineered cell and binds the endogenous cell surface molecule within the engineered cell.

18.-21. (canceled)

22. A method of treating cancer in a subject in need thereof comprising administering a therapeutically effective amount of a composition comprising the engineered immune cell of claim 1 to the subject, thereby treating cancer in a subject in need thereof.

23. (canceled)

24. The method of claim 22, wherein the cancer is a T-cell malignancy or a CD2 associated cancer.

25. The method of claim 24, wherein said T-cell malignancy or said CD2 associated cancer is selected from the group consisting of T cell leukemia T cell lymphoma, T-cell acute lymphoblastic leukemia (T-ALL), early T-cell progenitor acute lymphoblastic leukemia (ETP-ALL), T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, cutaneous T-cell lymphomas (CTCL), any subtype of CTCL, mycosis fungoides, Sézary syndrome, primary cutaneous gamma-delta T-cell lymphoma, a malignancy with the T lineage subsets of Non-Hodgkin's lymphoma (NHL), peripheral T-cell lymphoma (PTCL) not otherwise specified (PTCL-NOS), angioimmunoblastic T-cell lymphoma, and anaplastic large cell lymphoma.

26. The method of claim 22, wherein said administration is by any one selected from the group consisting of intravenous infusion, intra-arterial infusion, intraperitoneal infusion, direct injection into tumor and/or perfusion of tumor bed after surgery, implantation at a tumor site in an artificial scaffold, and intrathecal administration.

27. An expression vector composition comprising an expression vector comprising a polynucleotide encoding said CAR of claim 1.

28. An expression vector composition comprising one or more expression vectors comprises a polynucleotide for a guide RNA complementary to the human CD2 gene, and a polynucleotide encoding a Cas protein, wherein the polynucleotide for the guide RNA comprises any one selected from the group consisting of SEQ ID NOS:44-47.

29. (canceled)

30. A method for producing an engineered immune cell of claim 1, the method comprising: introducing the expression vector composition of claim 27, and the expression vector composition of claim 28 into an immune cell.

31.-32. (canceled)
